(19) United States
(12) Patent Application Publication
(10) Pub. No.: US 2021/0317522 A1
(43) Pub. Date: Oct. 14, 2021

(54) PHENOTYPIC AND MOLECULAR CHARACTERISATION OF SINGLE CELLS

(71) Applicant: GARVAN INSTITUTE OF MEDICAL RESEARCH, Darlinghurst, New South Wales (AU)

(72) Inventors: Alexander SWARBRICK, Darlinghurst, New South Wales (AU); Martin SMITH, Darlinghurst, New South Wales (AU); Christopher GOODNOW, Darlinghurst, New South Wales (AU); James FERGUSON, Darlinghurst, New South Wales (AU); Ghamdan AL-ERYANI, Darlinghurst, New South Wales (AU); Mandeep SINGH, Darlinghurst, New South Wales (AU); Shaun CARSWELL, Darlinghurst, New South Wales (AU); Katherine JACKSON, Darlinghurst, New South Wales (AU)

(73) Assignee: GARVAN INSTITUTE OF MEDICAL RESEARCH, Darlinghurst, New South Wales (AU)

(21) Appl. No.: 17/277,446

(22) PCT Filed: Feb. 8, 2019
(86) PCT No.: PCT/AU2019/050101

§ 371 Date: Mar. 18, 2021

(30) Foreign Application Priority Data
Sep. 21, 2018 (AU) 2018903546

(51) Int. Cl. C12Q 1/6869; G16B 30/10

(52) U.S. Cl. CPC C12Q 1/6869; G16B 30/10

(57) ABSTRACT

The present disclosure relates to an improved methodology for phenotyping and molecular characterisation of single cells using high-throughput and multiplexed targeted long-read single cell sequencing. In one particular example, the present disclosure relates to a methodology which combines targeted long-read sequencing with short-read based transcriptome profiling of barcoded single cell libraries generated by droplet-based partitioning for high throughput deep single cell profiling.

### RELATED APPLICATION DATA

The present application claims priority from Australian Provisional Application No. 2018903546 filed on 21 Sep. 2018, the full contents of which is incorporated herein by reference.

### TECHNICAL FIELD

The present disclosure relates to an improved methodology for phenotyping and molecular characterisation of single cells using high-throughput and multiplexed targeted long-read single cell sequencing. In one particular example, the present disclosure relates to a methodology which combines targeted long-read sequencing with short-read based transcriptome profiling of barcoded single cell libraries generated by droplet-based partitioning for high throughput deep single cell profiling.

### BACKGROUND

B and T lymphocytes recognise foreign and self-antigens through their antigen receptors which in turn govern their development, survival and activation. To establish a diverse repertoire of antigen-specific lymphocytes, the T Cell Receptor (TCR) and B Cell Receptor (BCR) are assembled from variable (V), diversity (D) and joining (J) gene segments in a somatic process known as V(D)J recombination (Bassing et al., (2002) Cell, 109(Suppl):545-55). Random addition or removal of nucleotides at the complementarity determining region 3 (CDR3), which adjoins V(D)J junctions, largely determines the specificity towards antigen. Due to the significant diversity of both the BCR and TCR repertoire, estimated at >10¹² (Calis and Rosenberg (2014) Trends in immunology, 35(12):581-90; Laydon et al., (2015) Biological sciences, 370(1675)), it is highly likely that two cells carrying the same antigen receptor sequence are clonally related and constitute a clonotype. As a result, when a B cell or T cell clone undergoes clonal expansion the identity of a BCR or TCR sequence serves as a unique clonal identifier or ‘clonal barcode’ and provides information on antigen specificity and clonal ancestry.

Sequencing the BCR or TCR of individual lymphocytes in parallel with their transcriptome provides high resolution insights into the adaptive immune response in a range of disease settings such as infectious disease, autoimmune disorders and cancer. A common approach to link paired antigen receptor sequences with gene expression profiles of single lymphocytes is through the use of the full-length scRNA-seq method SmartSeq2 (Picelli et al., (2013). Nature methods, 10(11):1096-8), where computational methods can reconstruct paired TCRαβ sequences or paired IgH and IgL sequences from Illumina short-reads (Afik et al., (2017) Nucleic acids research, 45(16):e148; Eltahla et al., (2016) Immunology and cell biology, 94(6):604-11; Stubbington et al., (2016) Nature methods, 13(4):329-32; Upadhyay et al., (2018) Genome medicine, 10(1):20). However, SmartSeq2 generally relies on plate- or well-based microfluidics and is therefore limited in the number of cells that can be processed, typically 10s to 100s. Additionally, a large number of sequencing reads are generally required to computationally reconstruct paired antigen receptors. As such, the cost per cell is relatively high ($50-$100 USD).

Recent technological advancements in high-throughput scRNA-seq methods allow thousands of cells to be captured and sequenced in a relatively short time frame and at a fraction of the cost (Ziegenhain et al., (2017) Molecular cell., 65(4):631-43.e4). Such methods rely on capture of polyadenylated (polyA) mRNA transcripts followed by cDNA synthesis, pooling, amplification, library construction and Illumina 3′ or 5′ cDNA sequencing. The combination of fragmentation and short-read sequencing fails to sufficiently sequence the V(D)J regions of rearranged TCR and BCR transcripts, which are located closer to the 5′ end of the transcript. Consequently, 3′-tag scRNA-seq platforms have limited application for determining clonotypic information from large numbers of lymphocytes. Recent advances in long read sequencing technologies present a potential solution to the shortcomings of short-read sequencing. Full-length cDNA reads can encompass the entire sequence of BCR and TCR transcripts, but typically suffer from higher error rates and lower sequencing depth than short read technologies.

Accordingly, there is a need for improved methodologies for high-throughput and multiplexed targeted long-read single cell sequencing, such as for use in cellular phenotyping.

### SUMMARY

The present disclosure is based, at least in part, on the recognition by the inventors that, although existing high-throughput single-cell RNA-seq (scRNA-seq) is a powerful tool for gene expression profiling of complex and heterogeneous biological systems, e.g., such as the immune system or populations of clonally diverse cancer cells, these existing methods only provide short-read sequence data from one end of a cDNA template, which is poorly suited to the investigation of gene-regulatory events such as alternative transcript isoforms or fusion genes, adaptive immune responses or somatic genome evolution. The inventors have therefore developed a method that combines targeted long-read sequencing with short-read based transcriptome profiling of barcoded single cell libraries generated by droplet-based partitioning. The present inventors have then used this methodology, termed Repertoire And Gene Expression by sequencing (RAGEseq), to accurately characterise T-cell (TCR) and B-cell (BCR) receptor transcripts and transcriptional profiles of more than 7138 lymphocytes sampled from the primary tumour and draining lymph node of a breast cancer patient. In doing so, the inventors were able to phenotype clonally-related lymphocytes between tissues, identify alternately-spliced BCR transcripts encoding receptors destined for secretion versus membrane localization, and reveal somatic hypermutation of BCRs. The inventors also used this methodology to analyse PTPRC splice variants encoding alternate isoforms of CD45, providing important information on whether lymphocytes are naive (CD45RA) or antigen-experienced (CD45RO). In addition to the valuable insight this may provide to an immunologist, it demonstrates the use of RAGE-seq to analyse splicing of a transcript which is less abundant than TCRs/BCRs. These results demonstrate that RAGE-seq is an accessible and cost-effective method for high throughput deep single cell profiling, applicable to a wide diversity of biological questions and challenges. These include tumour immunology, autoimmune disease, somatic mutation and clonal evolution in cancer and adaptive resistance to therapy.

Accordingly, in one example, the disclosure provides a method for high-throughput and multiplexed phenotyping and characterisation of single cells, said method comprising:
(a) preparing a library of nucleic acid molecules for one or more isolated single cells, wherein unique cell barcode sequences and unique molecular identifier (UMI) sequences are assigned and introduced to the nucleic acid molecules, optionally wherein unique tissue barcodes are also assigned and introduced to the nucleic acid molecules;
(b) dividing the library into at least two components comprising a first library component and a second library component;
(c) sequencing the first library component to produce a first set of sequence data;
(d) high-throughput molecular profiling of the first set of sequence data to identify sequences containing genetic, epigenetic and/or transcriptomic features that are capable of distinguishing between different cells;
(e) sequencing the second library component using a long read sequencing method to produce a second set of sequence data comprising long-read sequences;
(f) demultiplexing the second set of sequence data by comparing or matching the long-read sequences to distinguish between individual long-read sequences;
(g) inferring molecular profiles for the demultiplexed long-read sequences based on molecular profiles characterised for corresponding sequences in the first set of sequence data at (d), wherein corresponding sequences are identified using the UMIs, unique cell barcodes, unique tissue barcodes, or a combinations thereof
(h) assigning the long-read sequences into one or more groups based on information relating to one or more of tissue type, cell type, genes, sequences, and/or molecules of interest, and generating one or more contigs based on consensus sequences identified within the one or more groups; and
(i) undertaking molecular characterisation of the contigs.

In one example, unique tissue barcodes are assigned and introduced to the nucleic acid molecules at step (a) to enable subsequent pooling and multiplex analysis of nucleic acid molecules derived from more than one tissue type and/or sample e.g., including samples from different subjects.

The library of nucleic acid molecules may contain any nucleic acid molecule selected from the group consisting of cDNA, genomic DNA, barcodes, cellular RNA (e.g., such as messenger RNA (mRNA), transfer RNA (tRNA), ribosomal RNA (rRNA), small nuclear RNA (snRNA) and/or non-coding RNA (ncRNA)) and combinations thereof. In one example, the library of nucleic acid molecules comprises cDNA. In one example, the library of nucleic acid molecules comprises genomic DNA. In one example, the library of nucleic acid molecules comprises barcodes. In one example, the library of nucleic acid molecules comprises cellular RNA e.g., such as messenger RNA (mRNA), transfer RNA (tRNA), ribosomal RNA (rRNA), small nuclear RNA (snRNA) and/or non-coding RNA (ncRNA). In one example, the library of nucleic acid molecules comprises a mixture of cDNA, genomic DNA, barcodes and cellular RNA.

In on example, the method further comprises a single cell capture step prior to step (a). For example, single cell capture may be performed by one or more of the following means: a droplet-based microfluidics platform, a flow cytometry platform, a plate-based platform, a microwell-based platform or any combination thereof.

In one example, the method described herein further comprises a step of isolating the cells prior to the single cell capture step e.g., by disassociating tissue or bodily fluid into cellular components or by selection of one or more subsets of cells from said tissue or bodily fluid. The first library component may be sequenced using a short-read sequencing method and/or a long-read sequencing method. In one example, first library component is sequenced using a short-read sequencing method. In one example, the first library component is sequenced using a long-read sequencing method. In yet another example, the first library component is sequenced using both short and long-read sequencing methods.

In one example, the short-read sequencing method is a next generation sequencing (NGS) method selected from the group consisting of sequencing-by-hybridization, sequencing-by-synthesis, sequencing-by-ligation platform, ion semiconductor sequencing, combinatorial probe anchor synthesis, and combinations thereof. In one example, short-read sequencing may be performed using a sequencing-by-hybridization method. In one example, short-read sequencing may be performed using a sequencing-by-synthesis method. In one example, short-read sequencing may be performed using a sequencing-by-ligation method. In one example, short-read sequencing may be performed using ion semiconductor sequencing. In one example, short-read sequencing may be performed using a combinatorial probe anchor synthesis method. In another example, short-read sequencing may be performed using a combination of sequencing-by-hybridization, sequencing-by-synthesis, sequencing-by-ligation, ion semiconductor sequencing and combinatorial probe anchor synthesis methods.

In one example, the long-read sequencing method is a nanopore sequencing method, a single molecule real time (SMRT) sequencing method or a combination thereof. In one example, the long-read sequencing method is a nanopore sequencing method. In another example, the long-read sequencing method is a SMRT sequencing method. In yet another example, a combination of nanopore sequencing and SMRT sequencing is used.

In one example, the method comprises targeted enrichment of the first and/or second library components for sequences or features of interest prior to sequencing. In one example, the method comprises targeted enrichment of the second library component only. In another example, the method comprises targeted enrichment of the first and second library components. Targeted enrichment prior to sequencing may be performed using a hybridisation capture protocol. One exemplary hybridisation capture protocol relies on biotinylated hybridisation beads attached to capture probes which bind selectively to genetic, epigenetic or transcriptomic sequences or features or interest within the library component(s). However, other hybridisation capture method known in the art may also be used. Alternatively or in addition to the use of a hybridisation capture protocol, targeted enrichment of the library component(s) may comprise depleting unwanted sequences or features from the library component(s) prior to sequencing.

Alternatively, or in addition, the targeted enrichment may be performed in silico post-sequencing e.g., computational enrichment of sequence data based using filters. In accordance with this example, the in silico enrichment may preferentially select for sequences or features of interest in the first and/or second sets of sequence data. In another example, the in silico enrichment may deplete unwanted sequences or features from the first and/or second sets of sequence data.

In any one of the examples, targeted enrichment increases representation of sequences or features of interest with the first and/or second set of sequence data, particularly within the second set of sequence data. In accordance with any of the examples relating to targeted enrichment, the second set of sequence data may be enriched for T and/or B cell receptor sequences. However, a skilled person will appreciate that the sequence data may be enriched for any gene(s), sequence(s) and/or feature(s) of interest. For example, the sequence data may be enriched for immunological genes e.g., PTPRC encoding CD45. In one example, the molecular characterisation of the contigs is on the basis of one or more of the following: antigen receptor clonotyping, mutation analysis, somatic genome variation, alternative transcript splicing, fusion genes or chimeric transcripts, transcript isoform quantification and combinations thereof. However, depending on the enrichment performed (if any), the starting cell population(s) and/or the sequences of interest, the molecular characterisation may be based on other features of interest.

In accordance with one example in which targeted enrichment was performed for T and/or B cell receptor sequences, either before or after sequencing as described herein, molecular characterisation of the contigs may be performed using IgBlast.

Alternatively or in addition, the molecular characterisation of the contigs is on the basis of one or more of the following:
(i) information from step (d) inferred from the corresponding sequences in the first set of sequence data;
(ii) information relating to target enrichment for sequences or features of interest performed on the second library component prior to long-read sequencing and/or in silico following sequencing;
(iii) alignment of long-read sequences or contigs to an annotated reference sequences or genomes; and/or
(iv) information relating to the one or more of the unique cell barcodes, UMI sequences and/or unique tissue barcodes.

In one example, the method described herein comprises performing one or more filtering steps on the second set of sequence data to remove sequences which are below a desired length (e.g., <500 bases long), uninformative, erroneous and/or not of interest. Filtering may also involve removing adapter sequences added to sequences during preparation of the nucleic acid library. The filtering step may be performed on the second set of sequence data comprising long-read sequences at any time prior to de novo assembly into contigs. For example, the one or more filtering steps may be performed prior to demultiplexing the second set of sequence data. Alternatively, or in addition, one or more filtering steps may be performed after the demultiplexing step but prior to de novo assembly of long-read sequences into contigs.

In one example, demultiplexing of the second set of sequence data is supervised. For example, supervised demultiplexing may comprise comparing or matching the long-read sequences to the corresponding sequences in the first set of sequence data using the UMI, unique cell barcodes, optionally unique tissue barcodes or combinations thereof.

In another example, demultiplexing of the second set of sequence data is unsupervised. For example, unsupervised demultiplexing may comprise comparing the second set of sequence data to itself in a manner sufficient to identify commonalities in long-read sequences, or identifying sequence features selected from UMI, unique cell barcodes and/or unique tissue barcodes.

In yet another example, the demultiplexing of the second set of sequence data involves both supervised and unsupervised methods.

Assignment of the demultiplexed long-read sequences into one or more groups may comprise de novo assembly of long-read sequences, alignment to one or more reference sequences, multiple sequence alignments or other approach capable of grouping the long-read sequences e.g., based on tissue type, cell type and/or sequence type. Grouped long-read sequences may then be assembled and a contig formed on the basis of consensus sequence. In one example, de novo grouping and assembly of the demultiplexed long-read sequences into one or more contigs is performed using CANU software. In another example, grouping and assembly of the demultiplexed long-read sequences is performed by aligning the long sequence reads to sequences of interest corresponding to the enrichment targets of step (d) using the Minimap2 software, followed by multiple sequence alignment of aligned sequences using MAFFT software.

The method described herein may also comprise one or more consensus correction and/or polishing steps to correct errors in the long-read sequences and/or contigs and thereby improve the consensus sequence. The one or more correction and/or polishing steps may be performed after demultiplexing but before assembly into contigs. Alternatively, or in addition, the one or more correction and/or polishing steps may be performed on the contigs. For example, contig consensus correction may be performed using Minimap2 and/or RACON. For example, consensus polishing may be performed using Minimap2 and/or Nanopolish.

In some example, the method of the disclosure (RAGE-seq) may be performed in combination with one or more other analytical methods e.g., such as Cellular Indexing of Transcriptomes and Epitopes by sequencing (CITE-seq). Such a combinatorial multi-omic methodology may permit the simultaneous phenotyping of cellular populations using e.g., protein targets plus RNA with full length sequencing capacity. In accordance with an example in which the method of the disclosure is combined with CITE-seq, step (b) described hereinabove may comprise dividing the library into at least three components comprising the first library component, the second library component and a third library component, wherein the third library component comprises CITE-seq barcodes from antibodies.

The disclosure also provides a computer implemented method for phenotyping and characterising single cells using data obtained from high-throughput and multiplexed long-read single cell sequencing, said method comprising:
(a) receiving a first set of sequence data for a library of nucleic acid molecules generated for one or more isolated single cells, wherein each nucleic acid molecule in the library comprises a unique cell barcode sequence and unique molecular identifier (UMI) sequence, optionally wherein each nucleic acid molecule in the library also comprises a unique tissue barcode;
(b) high-throughput molecular profiling of the first set of sequence data by identifying sequences containing genetic, epigenetic and/or transcriptomic features that are capable of distinguishing between different cells;
(c) receiving a second set of sequence data for the library of nucleic acid molecules, said second set of sequence data comprising long-read sequences;

(d) demultiplexing the second set of sequence data to distinguish between individual long-read sequences;
(e) inferring molecular profiles for the demultiplexed long-read sequences based on molecular profiles characterised for corresponding sequences in the first set of sequence data at (b);
(f) assigning the long-read sequences into one or more groups based on information relating to one or more of tissue type, cell type, genes, sequences and/or molecules of interest, and generating one or more contigs based on consensus sequences identified within the one or more groups;
(g) undertaking molecular characterisation of the contigs; and
(h) generating user interface data comprising information relating to molecular characterisation of the contigs.

In one example, each nucleic acid molecule comprises a unique tissue barcode to enable pooling and deconvolution of sequence data for nucleic acid molecules derived from more than one tissue type and/or sample e.g., including samples from different subjects.

The library of nucleic acid molecules may contain any nucleic acid molecule selected from the group consisting of cDNA, genomic DNA, barcodes, cellular RNA (e.g., such as messenger RNA (mRNA), transfer RNA (tRNA), ribosomal RNA (rRNA), small nuclear RNA (snRNA) and/or non-coding RNA (ncRNA)) and combinations thereof. In one example, the library of nucleic acid molecules comprises cDNA. In one example, the library of nucleic acid molecules comprises genomic DNA. In one example, the library of nucleic acid molecules comprises barcodes. In one example, the library of nucleic acid molecules comprises cellular RNA e.g., such as messenger RNA (mRNA), transfer RNA (tRNA), ribosomal RNA (rRNA), small nuclear RNA (snRNA) and/or non-coding RNA (ncRNA). In one example, the library of nucleic acid molecules comprises a mixture of cDNA, genomic DNA, barcodes and cellular RNA.

The first set of sequence data may be generated by a short-read sequencing method and/or a long-read sequencing method. In one example, the first set of sequence data has been generated using a short-read sequencing method. In one example, the first set of sequence data has been using a long-read sequencing method. In yet another example, the first set of sequence data has been using both short and long-read sequencing methods.

In one example, the short-read sequencing method is a next generation sequencing (NGS) method selected from the group consisting of sequencing-by-hybridization, sequencing-by-synthesis, sequencing-by-ligation platform, combinatorial probe anchor synthesis and combinations thereof. In one example, short-read sequencing method is a sequencing-by-hybridization method. In one example, the short-read sequencing method is a sequencing-by-synthesis method. In one example, short-read sequencing method is a sequencing-by-ligation method. In one example, short-read sequencing method is a combinatorial probe anchor synthesis method. In another example, short-read sequencing method is a combination of sequencing-by-hybridization, sequencing-by-synthesis, sequencing-by-ligation, ion semiconductor sequencing and combinatorial probe anchor synthesis methods.

In one example, the second set of sequence data received has been generated by a nanopore sequencing method or a single molecule real time (SMRT) sequencing method. In one example, the second set of sequence data received has been generated using a nanopore sequencing method. In another example, the second set of sequence data received has been generated using a SMRT sequencing method. In yet another example, the second set of sequence data has been generated using a combination of nanopore sequencing and SMRT sequencing.

In one example, the first and/or second set of sequence data is enriched for genetic, epigenetic or transcriptomic sequences or features or interest. In one example, the second set of sequence data is enriched for genetic, epigenetic or transcriptomic sequences or features or interest. In another example, the first and second sets of sequence data are both enriched for genetic, epigenetic or transcriptomic sequences or features or interest. In accordance with an example in which the first and/or second sets of sequence data is/are enriched, targeted enrichment may have occurred prior to sequencing, following sequencing (e.g., by in silico means) or both. In one example, target enrichment comprises actively selecting for sequences or features or interest e.g., using a hybridisation capture protocol prior to sequencing and/or by in silico means following sequencing. Alternatively or in addition, target enrichment may comprise depleting unwanted sequences or features prior to sequencing and/or by in silico means following sequencing. In accordance with any of the examples relating to targeted enrichment, the targeted enrichment may be for T and/or B cell receptor sequences. However, a skilled person will appreciate that the sequence data may be enriched for any gene(s) and/or feature(s) of interest. For example, in another example the sequence data may be enriched for immunological genes e.g., PTPRC encoding CD45.

In one example, molecular characterisation of the contigs is on the basis of one or more of the following: antigen receptor clonotyping, mutation analysis, somatic genome variation, alternative transcript splicing, fusion genes or chimeric transcripts, transcript isoform quantification and combinations thereof. However, characterisation of contigs may be based on other features depending on whether the second set of sequence data comprising long-read sequences has been enriched for any particular sequences or features of interest, the starting cell population(s) and/or the sequences of interest.

In accordance with an example in which targeted enrichment was performed for T and/or B cell receptor sequences, molecular characterisation of the contigs may be performed using IgBlast.

Alternatively or in addition, the molecular characterisation of the contigs may be on the basis of one or more of the following:
(i) information relating to the molecular profile of long-read sequences inferred at (e);
(ii) information relating to target enrichment for sequences or features of interest;
(iii) alignment of long-read sequences or contigs to an annotated reference sequences or genomes; and/or
(iv) information relating to the one or more of the unique cell barcodes, UMI sequences and/or unique tissue barcodes.

In one example, the computer implemented method described herein comprises performing one or more filtering steps on the second set of sequence data to remove sequences which are below a desired length (e.g., <500 bases long), uninformative, erroneous and/or not of interest. Filtering may also involve removing adapter sequences added to sequences during preparation of cDNA libraries. The one or more filtering step may be performed on the second set of sequence data at any time prior to de novo assembly into contigs. For example, the one or more filtering steps may be performed prior to demultiplexing the second set of sequence data. Alternatively, or in addition, one or more filtering may be performed after the demultiplexing step but prior to de novo assembly of long-read sequences into contigs.

In one example, demultiplexing of the second set of sequence data is supervised. For example, supervised demultiplexing may comprise comparing or matching the long-read sequences to the corresponding sequences in the first set of sequence data using the UMI, unique cell barcodes, unique tissue barcodes or combinations thereof.

In another example, demultiplexing of the second set sequence data is unsupervised. For example, unsupervised demultiplexing may comprise comparing the second set of sequence data to itself in a manner sufficient to identify commonalities in the long-read sequences, or identifying sequence features selected from UMI, unique cell barcodes and/or unique tissue barcodes.

In yet another example, the demultiplexing of the second set of sequence data involves both supervised and unsupervised methods.

Assignment of the demultiplexed long-read sequences into one or more groups may comprise de novo assembly of long-read sequences, alignment to one or more reference sequences, multiple sequence alignments or other approach capable of grouping the long-read sequences e.g., based on tissue type, cell type and/or sequence type. Grouped long-read sequences may be assembled and a contig formed on the basis of consensus sequence. In one example, de novo grouping and assembly of the demultiplexed long-read sequences into one or more contigs is performed using CANU software. In another example, grouping and assembly of the demultiplexed long-read sequences is performed by aligning the long reads to sequences of interest corresponding to the enrichment targets of step (d) using the Minimap2 software, followed by multiple sequence alignment of aligned sequences using MAFFT software.

The computer implemented method described herein may also comprise one or more consensus correction and/or polishing steps to correct errors in the long-read sequences and/or contigs and thereby improve the consensus sequence. The one or more correction and/or polishing steps may be performed after demultiplexing but before assembly into contigs. Alternatively, or in addition, the one or more correction and/or polishing steps may be performed on the contigs. For example, contig consensus correction may be performed using Minimap2 and/or RACON. For example, consensus polishing may be performed using Minimap2 and/or Nanopolish.

### BRIEF DESCRIPTION OF DRAWINGS

FIG. 1 is a schematic of the RAGE-Seq experimental protocol and computational pipeline when used in the exemplary application of characterising B and T cells.

FIG. 2 provides a further schematic of the RAGE-Seq pipeline. As illustrated, droplet-based single cell capture is used to generate an initial barcoded cDNA library, which is split and simultaneously subjected to (i) short read sequencing for 3′ expression profiling and (ii) hybridisation capture using custom probes followed by long read sequencing. The short read sequencing is used to cluster cellular populations and to generate high-accuracy cell barcode sequences, which are then used to demultiplex the long read data. Demultiplexed long reads are then subjected to de novo assembly, error correction, and (in the case illustrated) clonotyping analysis, resolving the complete sequence of antigen receptors with single nucleotide accuracy.

FIG. 3 shows quality control measurements of calling somatic hypermutation in individual cells. (A) Nucleotide length of Ramos heavy chain (left) and light chain (right) V regions. The maximum length of the entire IGVH4-34 (left) or IGLV2-13 (right) gene is indicated. (B) Heatmap of the V regions of individual Jurkat cells encoding the TCRα (right) and TCRβ (light) chain. Each row represents an individual cell and each column a nucleotide position in the respective V gene. Light blue represents synonymous nucleotide substitutions while dark blue represents non-synonymous nucleotide substitutions, when compared to germline TRAV8-4 and TRBV12-3 sequences.

FIG. 4 shows RAGE-Seq cross-sequencing platform quality control measurements. (A) Reference CDR3, V and J gene segments that encode the TCRα and TCRβ chains of Jurkat or the immunoglobulin heavy and light chains of Ramos. For Ramos the most abundant heavy chain CDR3 (1123/1278 cells) and light chain CDR3 sequences (104/937) were chosen as the reference. (B) tSNE of key canonical gene expression markers used to identify cell type of cluster. CD3G was used to identify Jurkat cells, CD79B was used to identify Ramos cells and LYZ and CD14 were used to identify monocyte cells. (C) The relative enrichment of targeted capture of antigen receptor genes. On-target reads were determined by the percentage of total Nanopore or Illumina sequencing reads that align to TRA, TRB, IGH, IGL and IGK constant region genes. (d) Nanopore cell barcode recovery for Nanopore reads that are on-target. Mean on-target reads per cell type: Jurkat, 309; Ramos, 646; Monocyte, 1.49.

FIG. 5 provides data from short-read and targeted long-read single cell sequencing of immortalised B- and T-cell lines. (a) T-distributed stochastic neighbour embedding (tSNE) plot of short read sequencing data from 10× Chromium single-cell capture. Cell numbers: Jurkat=1,463; Ramos=2,000; monocytes=280 (b) Demultiplexing statistics for nanopore sequencing reads following targeted hybridisation capture of TCR and BCR baits. Each bar corresponds to the number of Nanopore reads per cell barcode identified with short read sequencing using exact sequence matching. Asterix indicates one cell with over 6,000 reads. Numbers next to each cell type correspond to the total number of cells with recovered barcodes. (c) Correlation between Illumina read counts and Nanopore read counts for T-cell receptor alpha constant gene (TRAC). Each point represents an individual Jurkat cell. (d) Nanopore read length distribution of de-multiplexing reads assigned to cell type compared to the length distribution of polished contigs that have been assigned productive clonotypes.

FIG. 6 shows Quality control measurements of antigen receptor assembly. (A) Mean number of contigs assembled per cell. Each bar corresponds to an individual cell. (B) The number of on-target Nanopore reads for Jurkat (left panel) or Ramos (right panel) grouped by the recovery of TCR chains or BCR chains, respectively. NR, no receptor. Only those TCR and BCR chains that match their reference V and J gene and contain in-frame CDR3 sequences and lack stop codons, termed a productive clonotype, were assigned. (D) The recovery of Jurkat cells assigned a TCRα chain (left panel) or Ramos cells assigned a Immunoglobulin heavy chain (right panel) as a function of the number of Illumina UMIs TRAC (Jurkat) or IGHM (Ramos) genes per cell. (E) Assignment of TCR chains to Jurkat cells or BCR chains to Ramos cells based on their V and J gene segment usage. Shown in each pie graph is the number of cells expressing the designated V and J genes. Only productive clonotypes are assigned. (F) tSNE plot of Jurkat, Ramos and monocyte cells (A) assigned TCR (top panel) or BCR (bottom panel) chains. Right panels show the total number of cells assigned different chains for each cell type. Doublets (n=136 cells) are not shown on the tSNE plots and were filtered out based on high gene count (see Methods). (G) Accuracy of CDR3 sequences of Jurkat cells at each stage of contig assembly and polishing. Shown are the number of cells assigned a CDR3 sequence that match the reference TRA or TRB CDR3. ‘Non-reference’ refers to a CDR3 sequence that does not match the reference Jurkat CDR3. ‘Non-productive’ refers to TCR chains with a CDR3 sequence that is out-of-frame or contains stop-codons and are usually filtered from the dataset. Only TCR chains that match the Jurkat reference V and J gene segments are assigned.

FIG. 7 shows data relating to validation of antigen receptor assembly. (a) Number of cells assigned productive TCRα and TCRβ clonotypes for Jurkat cells (n=1463) or productive IgH and IgL clonotypes for Ramos cells (n=2000). Clonotypes were assigned if they expressed the reference V and J gene combinations of Jurkat (TCRα: TRAV8-4-TRAJ3; TCRβ: TRBV12-3:TRBJ1-2) or Ramos (IgH: VH4-34-IGHJ6; IgL: IGLV2-14:IGLJ2). (b) CDR3 accuracy measured by the number of Jurkat cells with TCRα or TCRβ clonotypes that directly match their reference Jurkat CDR3 nucleotide sequences (referred to in FIG. 4). ‘Non-reference’ refers to a cell with a productive CDR3 sequence that does not match the reference. ‘Non-productive’ refers to clonotypes with a CDR3 sequence that is out-of-frame or contains stop-codons and are usually filtered from a dataset. Only cells with reference V and J gene combinations were analysed (referred to in FIG. 3). (c) Recovery of TCR and BCR clonotypes as a function of sequencing depth. Subsampling was performed on 200 Jurkat and 200 Ramos cells with >1000 reads. For Ramos, cells with the most common IgH and IgL CDR3 sequence (referred to in FIG. 4) were pre-selected. A chain is qualified as recovered if it contains its reference V and J genes. (d) Accuracy of the assembled CDR3 sequence versus the reference CDR3 in function of sequencing depth of sampling, as described in (c).

FIG. 8 shows tracking of somatic hypermutation in an immortalized B-cell line. (a) Inferred amino acid composition of the IgH and IgL V regions of Ramos cells assigned paired BCRs (n=615). Each row represents an individual cell. Blue rectangles represent non-germline amino acids, indicative of somatic hypermutation. On the right, a hierarchical clustering dendrogram of the concatenated IgL and IgH sequences is shown. (b) Network diagram of Ramos somatic hypermutation, where each node corresponds to a unique sequence and the edges correspond to the number of amino acid differences between them. The largest node in the centre is the predominant sequence in this Ramos cell line, which differs from the germline reference sequence. Diagram generated with Cytoscape.

FIG. 9 provides data demonstrating the use of RAGE-seq on a human lymph node. (a) tSNE plot associated with 3′ gene expression profiling of 6,027 lymph node cells captured on the 10× Chromium platform. Number of cells: B cell memory, 738; B cell naïve, 853; CD4 EM, 1069; CD4 CM1, 1096; CD4 CM 2, 226; TfH, 142; Treg, 740; CD8 CM, 487; CD8EF/NKT 405, 405; Plasmablast, 28; Innate-like, 144; Doublets, 86; Epithelial, 13. (b) Assignment of productive TCR and BCR clonotypes to each population identified in (a). (c) Characterisation of full-length IgH clonotypes assigned to individual Naïve (n=401) or Memory B cells (n=283) from the lymph node or Plasmablasts (P, n=15) from a matched tumour. Mutation rate (%) measures the percentage of the total number nucleotides in the V region mutated from germline. (d) Assignment of TCRγ and TCR clonotypes to the T cell compartments of the Lymph Node in (a). 92 T cells were assigned TCRγ clonotypes alone, 14 T cells were assigned TCR clonotypes alone and 11 T cells were assigned paired TCRγδ chains. (e) Visualisation of the tSNE plot in (a) for cells assigned paired BCR (n=689) or paired TCR (n=705) chains and amongst these cells those clones that are expanded. Clones were considered expanded if a paired TCR or BCR sequence was found in more than one cell. 13 T cell expanded clones and 13 B cell expanded clones were identified. Each clone was represented by 2 cells.

FIG. 10 provides additional measurements of RAGE-seq when used on a human lymph node. (A) tSNE of key canonical gene expression markers used to identify cell type of cluster. (B) The number of on-target nanopore reads for each cell population identified in the lymph node (top panel) and the recovery of cell barcodes for each cell within each cell population (bottom panel). The number of barcodes recovered is shown above the top panel. (C) The nucleotide length for assembled antigen receptor transcripts for each receptor chain identified across all cells in the lymph node. The overall nanopore sequencing read length distribution is shown as solid and the assembled contigs as dashed lines. (D) t-SNE plot of the assignment of TCR (top panel) and BCR (bottom panel) chains for each cell in the lymph node. (E) Mutation rate of the framework and complementarity regions of the heavy chain V regions that have been assigned to memory B cells. (F) TCRα and TCRb chain sequence of T cells assigned MAIT-associated TCRs. (G) Jaccard set similarity score of top 250 raw UMI gene counts across all B cells (top) and T cells (bottom) with shared (SAME) V(D)J sequences and those with dissimilar (DIFF) V(D)J sequences within each respective cell type cluster. Only cells assigned paired TCR or paired BCR chains were analyzed. Significance was calculated via the corrected Wilcoxon test.

FIG. 11 provides additional measurements of RAGE-seq when used on a tumour. (A) tSNE plot associated with 3′ gene expression profiling of a tumour sample captured on the 10× Chromium platform. (B) Assignment of TCR chains to each T population and BCR chains to each B cell population identified in (A). (C) tSNE plots of key canonical gene expression markers used to identify cell types in (A). (D) Cell cycle phase of all cells in Tumour with tSNE structure overlay. (E) Proportion of cell cycle phase of all CD8 T-cells cells and expanded clones within tumour. Top CD8 clone: “TRBV7-9 TRBJ2-3: ASSLAGRVPGDTQY” and second top CD8 clone: “TRBV7-9 TRBJ2-2: ASSLELTGELF”.

FIG. 12 shows repertoire analysis of patient matched lymph node and tumour: (a) tSNE generated from 3′ 10× Chromium capture of patient matched tumour and lymph node. Select lymphocytes found to express shared chains across both tissues are highlighted. (b) tSNE plot of tumour and lymph node integrated using the canonical correlation algorithm (CCA) of Seurat. Analysis of TCRβ and IgH recovered on all lymphocytes shows 7 shared clonotypes between tumour and lymph (30 cells). Of those, a shared nearest neighbour (SNN) graph placed 6 together within the same cluster (CD8 EFF) irrespective of tissue origin. (c) Differentially expressed genes performed on all cells within the CD8 cluster. The non-shared cluster was randomly down sampled to 50 cells solely for heatmap visualisation purposes (upper heatmap). Of the 1,328 differential expressed genes found (P<0.01, Wilcoxon signed-rank test), the top 65 were visualised using a dotplot for: shared clone vs none-shared cells, and for any clone that contains 3 or more cells (lower panels).

FIG. 13 shows the combination of RAGE-Seq and CITE-Seq to measure RNA and protein antigen abundance in the same cells, along with targeted capture sequencing. A metastatic triple negative breast cancer was stained with 87 barcoded antibodies against immune and tumour markers and immune checkpoint molecules. 3113 cells were captured using the 10× Chromium platform and subject to RAGE-Seq, capturing TCR, BCR and PTPRC (the gene encoding CD45). (A) Clustering on CITE-Seq data reveals novel cell population structures. (B) CD8 T cells clustering to 4 populations, including two CD103+ tissue resident populations with differing PD1 expression. (C) RAGE-Seq identified a clonally expanded T cell population with the CDR3 amino acid sequence ASRRPGTSAFGELF. This population resided within the CD103⁺PD1^{high} subset of tissue-resident cytotoxic T cells. (D) Analysis of PTPRC isoforms revealed that two of the clonally-expanded T cells express the PTPRC isoform encoding CD45RO (blue traces), as expected of tissue-resident cytotoxic T cells. This is validated by high expression of CD45RO by these cells, detected by CITE-Seq. In comparison a naive B cell expresses the PTPRC isoform encoding CD45RABC. This result is validated by detection of CD45RA protein expression by this cell, using CITE-Seq. (E) Demonstrates that targeted capture of genes, here PTPRC, rescues the drop-out. FIG. 14 shows T- and B-cell receptor chain recovery in function of the number of reads used to assemble the mRNA sequence of targeted genes following in-silico targeting. This figure is comparable to FIG. 7c, except an additional computational filtering step is performed on the long read sequencing data. Specifically, the long sequencing reads were first aligned to the nucleotide sequences corresponding to the genomic coordinates used to design the biochemical capture sequencing probes (‘baits’) using a long read aligner (minimap2). Only reads that align to these targets are assembled/or and polished. Here, the “.sam” output of the ‘baited’ alignments was then polished with Racon (“Algorithm X”) and Nanopolish (“Algorithm Y”) for the same input data as FIG. 7c, highlighting the improved efficiency of T-cell receptor chains (TCRA & TCRB) using the ‘baited’ assembly.

FIG. 15 shows the Nanopore sequencing statistics and indel correction. (A) Number of reads, read length and quality score for cell line, tumour and lymph node samples. Colour denotes each flowcell the run was performed on (R9.4 or R9.5 chemistry). (B) The total number of TCR chains assigned to Jurkat cells (n=1463) that carry indels in their TRAV or TRBV gene before indel correction (see Methods). TCR chains include those with non-productive CDR3 sequences. (C) The effect of indel correction on antigen receptor chain recovery. Shown are all productive BCR and TCR chains recovered across the cell line experiment (all Jurkat, Ramos and monocyte cells). ‘All V(D)J sequences’ refers to both productive and non-productive antigen receptor sequences.

### DETAILED DESCRIPTION

### General

Throughout this specification, unless specifically stated otherwise or the context requires otherwise, reference to a single step, feature, composition of matter, group of steps or group of features or compositions of matter shall be taken to encompass one and a plurality (i.e. one or more) of those steps, features, compositions of matter, groups of steps or groups of features or compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example or embodiment of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant DNA, recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

Throughout this specification, unless the context requires otherwise, the word “comprise”, or variations such as “comprises” or “comprising”, is understood to imply the inclusion of a stated step or element or integer or group of steps or elements or integers but not the exclusion of any other step or element or integer or group of elements or integers.

The term “and/or”, e.g., “X and/or Y” shall be understood to mean either “X and Y” or “X or Y” and shall be taken to provide explicit support for both meanings or for either meaning.

### Selected Definitions

As used herein, the term “unique molecular identifier”, “UMI” or similar refers to a nucleic acid sequence which can be assigned and introduced to an individual nucleic acid e.g., a cDNA molecule, and used to discriminate between individual nucleic acid molecules.

Similarly, terms such as “unique cell barcode sequence” and “unique tissue barcodes” will be understood to refer to unique sequences which, when introduced to a nucleic acid sequence e.g., such as during cDNA library construction, can be used to identify the cell or tissue (as appropriate) from which the nucleic acid sequences derives.

The term. “nucleic acid”, “nucleic acid molecule” and “polynucleotide” are used interchangeably herein to refer to a polymer having multiple nucleotide monomers. A nucleic acid can be single- or double-stranded; and can be DNA (e.g., cDNA or genomic DNA), RNA (e.g., mRNA, tRNA, rRNA, snRNA and/or ncRNA), or hybrid polymers (e.g., DNA/RNA). The term “nucleic acid” does not refer to any particular length of polymer. Rather, a nucleic acid may be any length e.g., greater than about 2 bases, greater than about 10 bases, greater than about 100 bases, greater than about 500 bases, greater than 1000 bases, greater than 10,000 bases, greater than 100,000 bases, greater than about 1,000,000 or more bases composed of nucleotides.

The term “sequencing,” as used herein, refers to a method by which the identity of a consecutive stretch of nucleotides within a nucleic acid molecule is identified. That is, the identify of individual nucleotides within the nucleic acid molecule are identified which collectively provide the sequence of the nucleic acid or a part thereof. A number of methods and platforms are known in the art for sequencing of nucleic acid molecules which are described herein. For the purpose of the present disclosure, these may be conveniently divided into short-read sequencing methods and long-read sequencing methods based on the capabilities of the various methods and sequencing chemistries.

As used herein, a “short-read sequencing method” shall be understood to mean sequencing methods which are capable of producing single reads of up to 1000 bases, such as from about 35 bases to about 1000 bases. However, typically short-read sequencing method produce reads of 500 bases or less. Exemplary short-read sequencing methods are described herein and include next generation sequencing methods such as sequencing-by-hybridization, sequencing-by-synthesis, sequencing-by-ligation platform, combinatorial probe anchor synthesis, and ion semiconductor sequencing. However, chain termination (Sanger sequencing) may also be used to produce short read sequences. In one particular example, a sequencing-by-synthesis method using the Illumina platform is used to produce short read sequences. It also follows that “short-read sequence data” is data comprising and/or relating to sequences of about 35 bases to about 1000 bases in length, and typically 500 bases or less.

Conversely, a long-read sequencing method shall be understood to mean a method capable of producing sequence reads in excess of 1000 bases. Exemplary long-read sequencing methods are described herein and include nanopore sequencing and single molecule real time (SMRT) sequencing. Of course, it will be appreciated that read length achieved using a long-read sequencing method is also dependent on preparation of the nucleic acid molecule library e.g., cDNA library, not just the sequencing platform. For example, if a library is produced with an average fragment length of about 500 bases, then the average length of reads obtained from such a library will not exceed that length. However, library preparation aside, it will be appreciated that long-read sequencing methods are capable of producing long-read sequences e.g., over 1 Mb. It also follows that “long-read sequence data” is data comprising and/or relating to sequences in excess of 1000 bases in length, such as for example, between 1000 bases and 500 kb. Preferably, “long-read sequences” in the context of the invention are full length cDNAs.

As used herein, the term “demultiplex”, demultiplexing” or similar shall be understood to mean a step or process of separating or dividing individual sequence reads within multiplexed sequence data comprising multiple sequences into separate sequence files based on an index sequence tag introduced to each sequence during construction of sequencing libraries.

The term “contigs” as used herein, refers to contiguous regions of DNA or RNA sequence. “Contigs” can be determined by any number methods known in the art, such as, by comparing sequencing reads for overlapping sequences, and/or by comparing sequencing reads against a databases of known sequences in order to identify which sequencing reads have a high probability of being contiguous.

As used herein, “single cell capture” will be understood to mean a process of isolating single cells from a population of cells.

As used herein, the term “targeted enrichment” shall be understood to refer to a process by which the relative representation of a particular species, category or type of nucleic acid molecule i.e., a target, within a population of different nucleic acid molecules is increased. As described herein, target enrichment may be achieved using a range of methods known in the art, such as hybridisation capture e.g., using biotinylated probes configured to hybridise to the target nucleic acid which can then be retrieved using a biotin ligand, and/or by a depletion method which removes unwanted nucleic acid species. Targeted enrichment may also be performed in silico following sequencing, either as an alternative or in combination with enrichment prior to or during sequencing.

As used herein, the term “assembling”, “assembly” or similar shall be understood to refer to a process comprising alignment of multiple sequences based on consensus regions in order to form a longer sequence. For example, assembly of multiple sequences may be for the purpose of reconstructing a longer sequence of which the multiple sequences are component parts.

As used herein, the term “clonotype” in the context of T and/or B cell sequences shall be understood to mean a specific antigen receptor sequence derived from V(D)J recombination during somatic genome rearrangement of T and B cells, which can be used to infer shared lymphocyte clonality, or evolutionary relatedness of lymphocytes. The specific sequence may contain mutations, such as introduced vis somatic hypermutation of B cells following their activation by antigen recognition, and therefore the most similar germline V(D)J sequence may be used to define the clonotype of a mutated V(D)J sequence. Following V(D)J recombination, it is extremely unlikely that two cells descended from different lymphocytes will carry the same antigen receptor sequence or ‘clonotype’.

### Methods

A method for high-throughput and multiplexed phenotyping and characterisation of single cells, said method comprising:
(a) preparing a library of nucleic acid molecules for one or more isolated single cells, wherein unique cell barcode sequences and unique molecular identifier (UMI) sequences are assigned and introduced to the nucleic acid molecules, optionally wherein unique tissue barcodes are also assigned and introduced to the nucleic acid molecules;
(b) dividing the library into at least two components comprising a first library component and a second library component;
(c) sequencing the first library component to produce a first set of sequence data;
(d) high-throughput molecular profiling of the first set of sequence data to identify sequences containing genetic, epigenetic and/or transcriptomic features that are capable of distinguishing between different cells;
(e) sequencing the second library component using a long read sequencing method to produce a second set of sequence data comprising long-read sequences; demultiplexing the second set of sequence data to distinguish between individual long-read sequences;
(g) inferring molecular profiles for the demultiplexed long-read sequences based on molecular profiles characterised for corresponding sequences in the first set of sequence data at (d), wherein corresponding sequences are identified using the UMIs, unique cell barcodes, unique tissue barcodes, or a combinations thereof
(h) assigning the long-read sequences into one or more groups based on information relating to one or more of tissue type, cell type, genes, sequences and/or molecules of interest, and generating one or more contigs based on consensus sequences identified within the one or more groups; and
(i) undertaking molecular characterisation of the contigs.

In one example, unique tissue barcodes are assigned and introduced to the nucleic acid molecules at step (a) to enable subsequent pooling and multiplex analysis of nucleic acid molecules derived from more than one tissue type and/or sample e.g., including samples from different subjects.

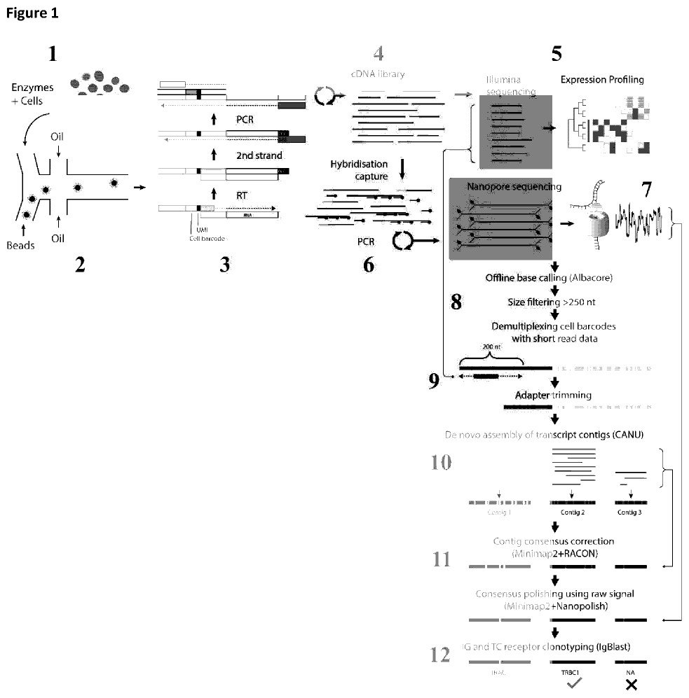

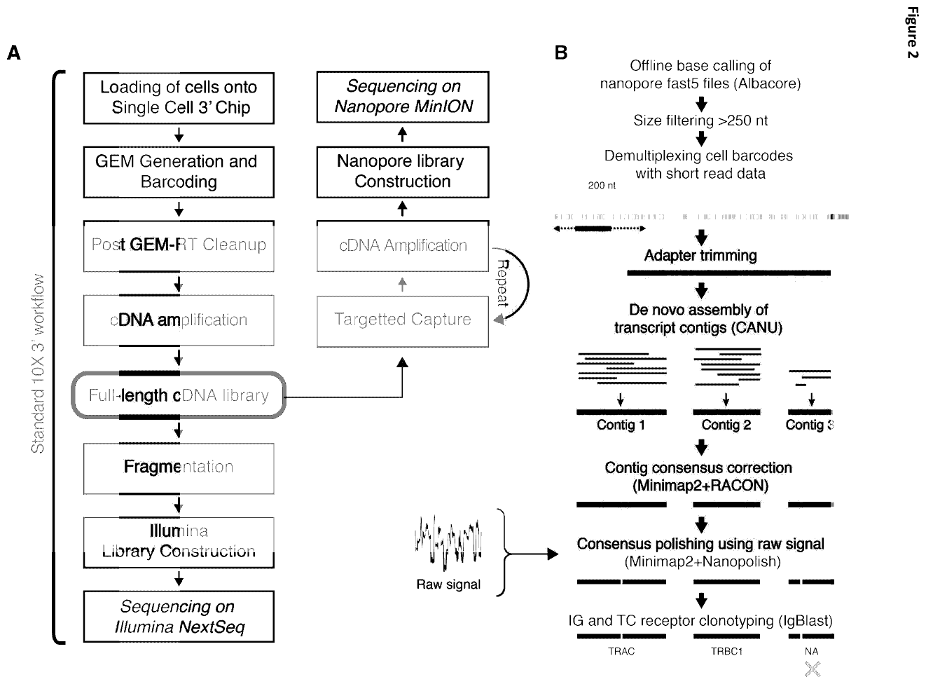

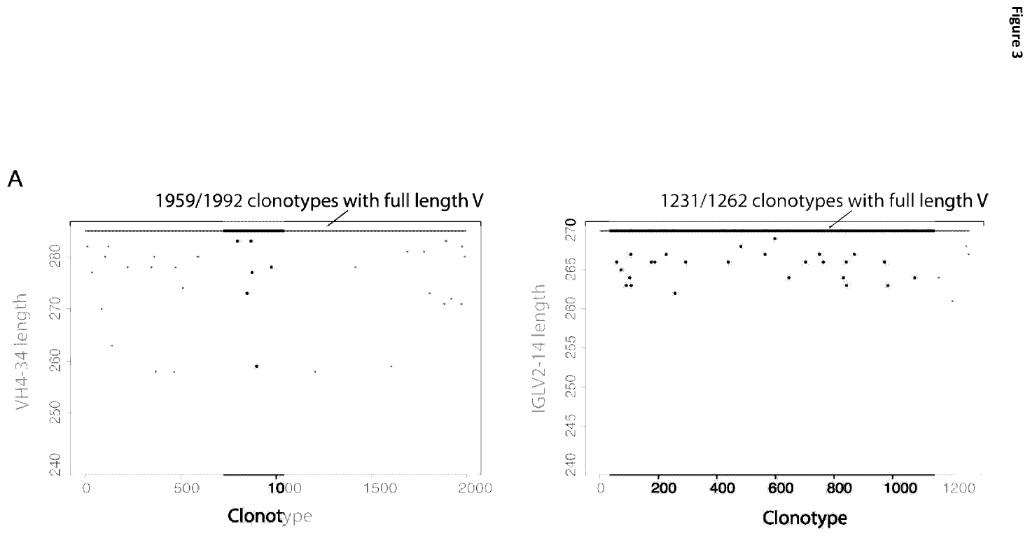

One exemplary method of the disclosure comprising steps (a) to (i) above is conveniently illustrated in FIG. 1 which will be referred to below.

Prior to steps (a) to (i) above, a single cell capture step may also be performed. However, the method may also be conveniently performed on cells previously isolated. Exemplary methods for single cell capture which may be employed in the method of the disclosure include a droplet-based microfluidics platform, a flow cytometry platform, a plate-based platform, microwell-based platform or any combination thereof. However, any single cell capture method known in the art may be used and is contemplated herein. In one particular example, a droplet-based microfluidics platform is used for single cell capture, as illustrated in FIG. 1. The method described herein may also comprise a step of isolating the cells e.g., from tissue, bodily fluid or cell culture, prior to the single cell capture step. Isolating cells may involve disassociating tissue or bodily fluid into cellular components or selection of one or more subsets of cells from said tissue, bodily fluid or cell culture. The tissue may be any tissue containing cells. Likewise, the bodily fluid may be any bodily fluid containing cells. The tissue, bodily fluid or cell culture may comprise healthy and/or cancerous cells. In one example the tissue, bodily fluid or cell culture comprises cancerous cells. In one example, the tissue, bodily fluid or cell culture comprises immune cells.

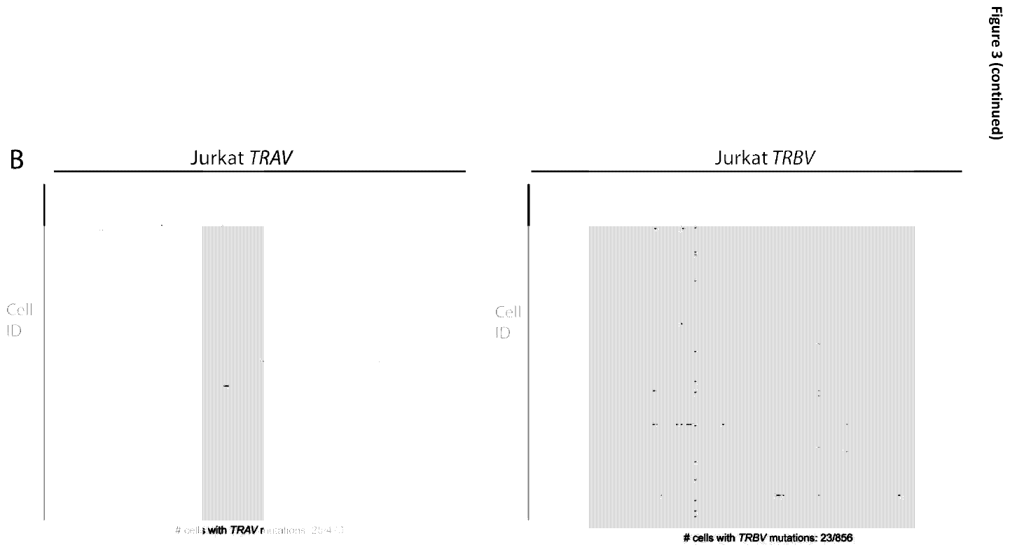

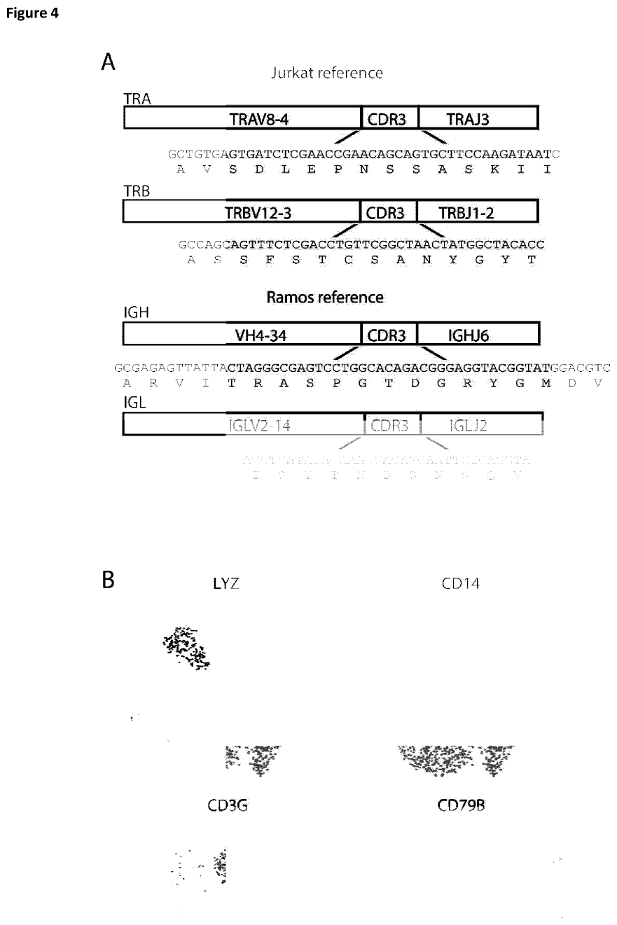

As described herein, the library of nucleic acid molecules may contain any nucleic acid molecule type, such as selected from the group consisting of cDNA, genomic DNA, barcodes, cellular RNA (e.g., mRNA, tRNA, rRNA, snRNA and/or ncRNA) and combinations thereof. However, in one example the method is performed with cDNA molecules, as illustrated in FIG. 1. In accordance with this example, full length cDNA libraries may be prepared at step (a) using any appropriate method known in the art. For example, methods of producing full length cDNA libraries are described in U.S. Pat. No. 6,197,554, Trombetta et al., (2014) Current Protocols in Molecular Biology, 107:4.22.1-4.22.17, Pan et al., (2013) PNAS, 110(2):594-599 and Cartolano et al., (2016) PLoS One, 11(6):e0157779, the contents of which are incorporated by reference herein. During the preparation of cDNA libraries from captured single cells, cell-specific tags, sequence-specific (also referred to as unique molecular identifiers (UMI's)) and tissue-specific tags can be introduced to cDNA or RNA molecules, as appropriate, in a manner that distinguishes individual molecules, cells, or samples that results in a library of molecules. Any sequence tags or barcodes which are sufficiently unique may be employed. Methods for introducing such tags (or barcodes) into cDNA libraries are known in the art and contemplated herein, such as described in Haque et al., (2017) Genome Medicine, 9:75 and elsewhere in the art. In one particular example, the full length barcoded cDNA library is prepared using the 10× Genomics platform (illustrated in FIG. 2).

As described herein and illustrated in FIG. 1, the resulting cDNA library is divided into two components: a first library component and a second library component. The first component is then subjected to high-throughput molecular profiling to characterise genetic, epigenetic, and/or transcriptomic features that are capable of distinguishing differences between single cells. As illustrated in step (5) of FIG. 1, this involve sequencing the first library component which may be achieved using a short-read sequencing method e.g., a next generation sequencing (NGS) method. However, the first library component may equally be sequenced using a long-read sequencing method as described herein.

There are a number of sequencing technologies available which may be employed for the short-read sequencing, such as, for example, the sequencing-by-hybridization platform from Affymetrix Inc. (Sunnyvale, Calif.), the sequencing-by-synthesis platforms from 454 Life Sciences (Bradford, Conn.), Illumina/Solexa (San Diego, Calif.) and Helicos Biosciences (Cambridge, Mass.), the sequencing-by-ligation platform from Applied Biosystems (Foster City, Calif.), ion semiconductor sequencing (also referred to as Ion Torrent sequencing) from ThermoFisher Scientific, and combinatorial probe anchor synthesis (cPAS) using the MGI Tech Platforms from BGI (China). However, other platforms are available and may be used. Furthermore, combinations of these platforms may be employed to generate the first set of sequencing data. In one particular example, sequencing is performed using a sequencing-by-synthesis method e.g., such as exemplified in FIG. 1. Once generated, computational analysis may be performed on the first set of sequence data to reveal discriminative features of the sequence population through molecular profiling e.g., discriminative features may include, but are not limited to genetic, epigenetic and/or transcriptomic features capable of distinguishing between single cells.

The second library component is subjected to long-read sequencing e.g., this may be performed in parallel to the sequencing of the first library component described above. Any platform known in the art capable of sequencing reads in excess of 1000 nucleotides in length is contemplated and may be employed. However, exemplary long-read sequencing methods for use in the method described herein include nanopore sequencing developed for example, by Oxford Nanopore Technologies and single molecule real time (SMRT) sequencing (SMRT™ technology of Pacific Biosciences). In one example, the long-read sequencing method employed is a nanopore sequencing method. In another example, the long-read sequencing method is a SMRT sequencing method. In yet another example, a combination of nanopore sequencing and SMRT sequencing is used.

As illustrated in FIG. 1 at (6), the method of the disclosure may also comprise a targeted enrichment step whereby the second library component is enriched for sequences or features of interest prior to performance of the long-read sequencing. This enrichment ensures that the sequences of interest are sequenced and represented in the resulting data set. Any method for targeted enrichment of nucleic acid libraries known in the art mat be used. For example, the targeted enrichment may be performed using well known hybridisation capture protocols. One exemplary hybridisation capture protocol relies on biotinylated hybridisation beads attached to capture probes which bind selectively to genetic, epigenetic or transcriptomic sequences or features of interest within the second library component. However, any hybridisation capture method known in the art could be used and is contemplated e.g., such as described in US20150211047 A1, U.S. Pat. No. 9,567,632, US20150141258 A1, and Dapprich et al., (2016) BMC Genomics, 17:486, the contents of each of which are incorporated herein.

Alternatively or in addition to the use of hybridisation capture of sequences or features of interest, targeted enrichment may comprise depleting unwanted sequences or features from the long-read component.

In accordance with any of the examples relating to targeted enrichment, the long-read component may be enriched for T and/or B cell receptor sequences.

It is also contemplated that target enrichment may be performed on the resulting sequence data i.e., by in silico mean. That is, one or more in silico steps may be performed to enrich the second set of sequence data for sequences of interest e.g., by actively selecting for sequences of interest or depleting the data set of unwanted sequences. It is contemplated that an in silico target enrichment may be performed in addition to target enrichment of the second library component. However, it is possible that only in silico enrichment is performed.

In addition to the target enrichment performed on the second library component and/or the second set of sequence data, equivalent target enrichment steps may also be performed on the first library component and/or first set of sequence data. Thus, in some example, both the first and second sets of sequence data are enriched for target sequences.

As illustrated in FIG. 1, the method described herein may optionally comprise one or more filtering steps, wherein the second set of sequence data (i.e., comprising long-read sequences) is computationally filtered to remove sequences which are, for example, below a desired length (e.g., <500 bases long), uninformative, erroneous and/or not of interest. This may assist in enriching the second set of sequence data for sequences of interest. Appropriate programs for computational filtering will be known to a person skilled in the art. Filtering may also involve removing adapter sequences which were added during preparation of nucleic acid molecule libraries e.g., cDNA libraries. The filtering step(s) may be performed on the second set of sequence data at any time prior to de novo assembly into contigs. For example, as illustrated in FIG. 1, a filtering step may be performed prior to demultiplexing the second set of sequence data. However, a filtering step may equally be performed after the demultiplexing step but prior to de novo assembly of long-read sequences into contigs (either as an alternative or additional filtering step).

As described herein and illustrated at (9) of FIG. 1, the method involves a demultiplexing step in which unique cell barcode and UMI sequences, and optionally unique tissue barcodes (if applicable), assigned and introduced to the cDNA molecules during cDNA library construction, are identified in the long-read sequences to characterise the respective long-read sequences. Demultiplexing in accordance with the method described herein also involves comparing or matching the sequences within the first set of sequence data to the corresponding long-read sequences using the UMI and unique cell barcodes and optionally unique tissue barcodes. Thus, discriminative features of sequences within the first set of sequence data identified through molecular profiling at step (d) may be inferred for the corresponding, matched long-read sequences. These discriminative features may include, but are not limited to genetic, epigenetic and/or transcriptomic features capable of distinguishing between single cells. Matching of corresponding sequences in the first and second sets of sequence data can also be used as a further filter to identify and correct potential errors in the long-read sequences.

The demultiplexing of the second set of sequence data may be performed in a supervised fashion e.g., by matching or comparing the long-read sequences directly, allowing errors or not. Alternatively, an unsupervised method may be employed to demultiplex the second set of sequence data, such as by comparing the long-read sequence data to itself in a manner sufficient to identify commonalities in long-read sequences or data that is associated with discriminating sequence features e.g., UMIs, cell barcodes, tissue barcodes etc.

Once the second set of sequence data is demultiplexed, molecular profile information relevant to the individual long-read sequences can be inferred based on molecular profiles characterised for corresponding sequences in the first set of sequence data at (d), wherein corresponding sequences from the first and second sets of sequence data are identified using the UMIs, unique cell barcodes, unique tissue barcodes, or a combinations thereof. The individual long-read sequences can then be organised into one or more groups based on information commonly assigned to each sequence e.g., information relating to cell type, tissue type, genes, sequences, molecules and other discriminative features described herein. Organisation of the demultiplexed long-read sequences into one or more groups may comprise de novo assembly of long-read sequences, alignment of long-read sequences to one or more reference sequences, multiple sequence alignments or other approach capable of grouping the long-read sequences e.g., based on tissue type, cell type and/or sequence type. Within each group, long-read sequences can then be assembled and one or more contigs produced based on consensus sequence. Any suitable software known in the art for long-read assembly may be employed. In particular one example, de novo grouping and assembly of the demultiplexed long-read sequences into one or more contigs is performed using CANU software (Koren et al., (2017) Genome Research, 27:1-15. In accordance with an example in which 10× Genomics platform is used to prepare barcoded cDNA libraries, the GemCode may also be used to perform de novo assembly of grouped long-read sequences into contigs. This step is illustrated at (10) of FIG. 1. In another example, grouping and assembly of the demultiplexed long-read sequences is performed by aligning the long sequence reads to sequences of interest corresponding to the enrichment targets of step (d) using the Minimap2 software, followed by multiple sequence alignment of aligned sequences using MAFFT software.

As illustrated at step (11) of FIG. 1, the method described herein may also optionally comprise one or more correction and/or polishing steps to correct errors in the long-read sequences and/or contigs and thereby improve the consensus sequence. For example, contig consensus correction may be performed using Minimap2 and/or RACON. For example, consensus polishing may be performed using Minimap2 and/or Nanopolish. However, any software programs known to be useful for correction and/or polishing of sequences may be employed and are contemplated for use herein.

Molecular characterisation of the contigs may then be undertaken. In one example, the molecular characterisation of the contigs comprises characterisation of contigs on the basis of one or more of the following: antigen receptor clonotyping, mutation analysis, somatic genome variation, alternative transcript splicing, fusion genes or chimeric transcripts, transcript isoform quantification and combinations thereof. In one example, molecular characterisation of the contigs may be performed using IgBlast. IgBlast may be particularly useful in circumstances in which the second set of sequence data has been enriched for T and/or B cell receptor sequences, either prior to long-read sequencing (as illustrated in FIG. 1) or in silico. However, depending on the enrichment performed (if any), the starting cell population(s) and/or the sequences of interest, characterisation may be based on other features of interest and using appropriate software.

Molecular characterisation of the contigs may also be based on one or more of the following:
(i) information from step (d) inferred from the corresponding sequences in the first set of sequence data;
(ii) information relating to target enrichment for sequences or features of interest performed on the second library component prior to long-read sequencing and/or in silico following sequencing;
(iii) alignment of long-read sequences or contigs to an annotated reference sequences or genomes; and/or
(iv) information relating to the one or more of the unique cell barcodes, UMI sequences and/or unique tissue barcodes.

### Computer-Implemented Methods

The disclosure also provides a computer implemented method for phenotyping and characterising single cells using data obtained from high-throughput and multiplexed long-read single cell sequencing, comprising:
(a) receiving a first set of sequence data for a library of nucleic acid molecules generated for one or more isolated single cells, wherein each nucleic acid molecule in the library comprises a unique cell barcode sequence and unique molecular identifier (UMI) sequence, optionally wherein each nucleic acid molecule in the library also comprises a unique tissue barcode;
(b) high-throughput molecular profiling of the first set of sequence data by identifying sequences containing genetic, epigenetic and/or transcriptomic features that are capable of distinguishing between different cells;
(c) receiving a second set of sequence data for the library of nucleic acid molecules, said second set of sequence data comprising long-read sequences;
(d) demultiplexing the second set of sequence data by comparing or matching the long-read sequences to corresponding sequences in the first set of sequence data using the UMI and unique cell barcodes and optionally unique tissue barcodes;
(e) inferring molecular profiles for the demultiplexed long-read sequences based on molecular profiles characterised for corresponding sequences in the first set of sequence data at (b);
(f) assigning the long-read sequences into one or more groups based on information relating to one or more of tissue type, cell type, unique molecules, genes, sequences and/or molecules of interest, and generating one or more contigs based on consensus sequences identified within the one or more groups;
(g) undertaking molecular characterisation of the contigs; and
(h) generating user interface data comprising information relating to molecular characterisation of the contigs.

In one example, each nucleic acid molecule comprises a unique tissue barcode to enable pooling and deconvolution of sequence data for nucleic acid molecules derived from more than one tissue type and/or sample e.g., including samples from different subjects.

As described herein, the library of nucleic acid molecules may contain any nucleic acid molecule selected from the group consisting of cDNA, genomic DNA, barcodes, cellular RNA (e.g., such as messenger RNA (mRNA), transfer RNA (tRNA), ribosomal RNA (rRNA), small nuclear RNA (snRNA) and/or non-coding RNA (ncRNA)) and combinations thereof. In one example, the library of nucleic acid molecules comprises cDNA. In one example, the library of nucleic acid molecules comprises genomic DNA. In one example, the library of nucleic acid molecules comprises barcodes. In one example, the library of nucleic acid molecules comprises cellular RNA e.g., such as messenger RNA (mRNA), transfer RNA (tRNA), ribosomal RNA (rRNA), small nuclear RNA (snRNA) and/or non-coding RNA (ncRNA). In one example, the library of nucleic acid molecules comprises a mixture of cDNA, genomic DNA, barcodes and cellular RNA.

The first set of sequence data may be generated by a short-read sequencing method and/or a long-read sequencing method. In one example, the first set of sequence data has been generated using a short-read sequencing method. In one example, the first set of sequence data has been using a long-read sequencing method. In yet another example, the first set of sequence data has been using both short and long-read sequencing methods. Short-read sequencing methods have been described herein and shall be taken to apply mutatis mutandis to each and every example describing computer implemented methods In one particular example, the first set of sequence data was generated by a method employing a sequencing-by-synthesis method e.g., as illustrated in FIG. 1.

Computational analyses may be performed on the first set of sequence data to reveal discriminative features of the sequence population through single cell molecular profiling e.g., discriminative features may include, but are not limited to genetic, epigenetic and/or transcriptomic features capable of distinguishing between single cells.

As described herein, the second set of sequence data received may be generated by any platform known in the art capable of consistently sequencing and transmitting reads in excess of 1000 nucleotides in length. Exemplary long-read sequencing technologies have already been described herein and shall be taken to apply mutatis mutandis to each and every example describing computer implemented methods. In one particular example, the second set of sequence data received has been generated using a nanopore sequencing method.

The second set of sequence data may be enriched for genetic, epigenetic or transcriptomic sequences or features or interest i.e., to ensure that sequences of interest are appropriately represented in the data. The first set of sequence data may also be enriched for the genetic, epigenetic or transcriptomic sequences or features or interest i.e., to ensure that sequences of interest are appropriately represented in the data. In one example, the first and/or second sequence data sets may have been produced using a method whereby target enrichment was performed prior to sequencing. Alternatively, or in addition, the first and/or second sequence data sets may have been produced using a method whereby target enrichment was performed post sequencing. In the case of the latter, the computer implemented method may comprise performing one of more in silico steps to enrich the first and/or second sets of sequence data for sequences or features of interest. Examples of target enrichment have been described herein and shall be taken to apply mutatis mutandis to each and every example describing computer implemented methods. In one particular example, the second set of sequence data is enriched for T and/or B cell receptor sequences.

The computer implemented method may comprises performing one or more filtering step on the second set of sequence data to remove sequences which are, for example, of undesired length (e.g., <500 bases long), uninformative, erroneous and/or not of interest. This may also assist in enriching the second set of sequence data for sequences of interest. Appropriate programs for computational filtering of sequence data will be known to a person skilled in the art. Filtering may also involve removing adapter sequences which were added during preparation of nucleic acid molecules libraries e.g., cDNA libraries. The filtering step may be performed on the second set of sequence data at any time prior to de novo assembly into contigs. For example, as illustrated in FIG. 1, the filtering step may be performed prior to demultiplexing the second set of sequence data. However, the filtering step may equally be performed after the demultiplexing step but prior to de novo assembly of long-read sequences into contigs.

The computer implemented method described herein involves a demultiplexing step in which the second set of sequence data is separated into its component sequences. Further, unique cell barcode and UMI sequences, and optionally unique tissue barcodes (if applicable), assigned and introduced to the nucleic acid molecules e.g., cDNA molecules, during library construction, are identified in the long-read sequences to characterise the respective long-read sequences. Demultiplexing also involves comparing or matching the long-read sequences to the corresponding sequences in the first set of sequence data using the UMI and unique cell barcodes and optionally unique tissue barcodes. Thus, discriminative features of the sequences in the first set of sequence data identified through molecular profiling at step (d) may be inferred for the corresponding, matched long-read sequences. These discriminative features may include, but are not limited to genetic, epigenetic and/or transcriptomic features capable of distinguishing between single cells. Matching of long-read sequences to corresponding sequences in the first set of sequence data can also be used as a further filter to identify and correct potential errors in the long-read sequences.

The demultiplexing of the long-read sequence data may be performed in a supervised fashion e.g., by matching or comparing the long-read sequences directly, allowing errors or not. Alternatively, an unsupervised method may be employed to demultiplex the long-read sequences, such as by comparing the second set of sequence data to itself in a manner sufficient to identify commonalities in long-read sequences or data that is associated with discriminating sequence features e.g., UMIs, cell barcodes, tissue barcodes etc.

Once the second set of sequence data is demultiplexed, molecular profile information relevant to the individual long-read sequences can be inferred based on molecular profiles characterised for corresponding sequences in the first set of sequence data at (b), wherein corresponding sequences from the first and second sets of sequence data are identified using the UMIs, unique cell barcodes, unique tissue barcodes, or a combinations thereof. The individual long-read sequences can then be organised into one or more groups based on information commonly assigned to each sequence at the demultiplexing step e.g., information relating to cell type, tissue type, unique molecules, sequence of interest and other discriminative features described herein. Organisation of the demultiplexed long-read sequences into one or more groups may comprise de novo assembly of long-read sequences, alignment of long-read sequences to one or more reference sequences, multiple sequence alignments or other approach capable of grouping the long-read sequences e.g., based on tissue type, cell type and/or sequence type. Within each group, long-read sequences can then be assembled and one or more contigs produced based on consensus sequence. Any suitable software known in the art for long-read assembly may be employed. In particular one example, de novo grouping and assembly of the demultiplexed long-read sequences into one or more contigs is performed using CANU software (Koren et al., (2017) Genome Research, 27:1-15. In accordance with an example in which 10× Genomics platform is used to prepare barcoded cDNA libraries, the GemCode may also be used to perform de novo assembly of grouped long-read sequences into contigs. This step is illustrated at (10) of FIG. 1. In another example, grouping and assembly of the demultiplexed long-read sequences is performed by aligning the long sequence reads to sequences of interest corresponding to the enrichment targets of step (d) using the Minimap2 software, followed by multiple sequence alignment of aligned sequences using MAFFT software.

The computer implemented method may also optionally comprise one or more correction and/or polishing steps to correct errors in the long-read sequences and/or contigs and thereby improve the consensus sequence. As described herein, the one or more correction and/or polishing steps may be performed at multiple stages of the method e.g., prior to demultiplexing, post-demultiplexing, post contig assembly. For example, contig consensus correction may be performed using Minimap2 and/or RACON. For example, consensus polishing may be performed using Minimap2 and/or Nanopolish. However, any software programs known to be useful for correction and/or polishing of sequences may be employed and are contemplated for use herein.

Molecular characterisation of the contigs may then be undertaken. In one example, the molecular characterisation of the contigs comprises characterisation of contigs on the basis of one or more of the following: antigen receptor clonotyping, mutation analysis, somatic genome variation, alternative transcript splicing, fusion genes or chimeric transcripts, transcript isoform quantification and combinations thereof. In one example, molecular characterisation of the contigs may be performed using IgBlast. IgBlast may be particularly useful in circumstances where an enrichment has been performed for T and/or B cell receptor sequences (as illustrated in FIG. 1). However, characterisation may be based on other features of interest and using appropriate software depending on the enrichment performed (if any), the starting cell population(s) and/or the sequences of interest.

Molecular characterisation of the contigs may also be based on one or more of the following:
(i) information relating to the molecular profile of long-read sequences inferred at (e);
(ii) information relating to target enrichment for sequences or features of interest;
(iii) alignment of long-read sequences or contigs to an annotated reference sequences or genomes; and/or
(iv) information relating to the one or more of the unique cell barcodes, UMI sequences and/or unique tissue barcodes.

### EXAMPLES

### Example 1—High-Throughput Targeted Long-Read Single Cell Sequencing Reveals the Clonal and Transcriptional Landscape of Lymphocytes

This example describes a rapid high-throughput method for sequencing full length transcripts using targeted capture and Oxford nanopore sequencing, and linking this with short-read transcriptome protein epitope profiling at single cell resolution. This novel method, termed Repertoire and Gene Expression by sequencing (RAGE-seq), may be applied to high-throughput droplet-based scRNA-seq workflows to accurately pair gene expression profiles with targeted full length cDNA sequences from a large number of cells.

In addition to describing the method, this example demonstrates the power of RAGE-seq by generating full transcriptome and full length sequence for antigen receptors and PTPRC (encoding CD45) from thousands of human tumour-associated lymphocytes. Using de novo assembly of nanopore reads, clonotype sequences were recovered at high accuracy and sensitivity, including the accurate calling of somatic mutations from full-length IgH and IgL chains allowing for the inference of B cell clonal evolution. Furthermore, PTPRC splice variants encoding alternate isoforms of CD45 were determined by targeted capture and long-read sequencing, providing important information on whether lymphocytes were naive (CD45RA) or antigen-experienced (CD45RO).

Finally, it is shown that RAGE-Seq is uniquely compatible with Cellular Indexing of Transcriptomes and Epitopes by sequencing (CITE-seq) (doi:10.1038/nMeth.4380), a remarkable new method that permits simultaneous measurement of transcriptomes and protein epitopes at single cell resolution. The combination of RAGE-Seq with CITE-Seq affords extremely high resolution multi-omic analysis of cellular phenotype and transcriptional output.

### 1.1 STRATEGY FOR RAGE-SEQ

Droplet-microfluidics form one of the most commonly used high-throughput single-cell RNA-sequencing methods due to their fast encapsulation of a large number of individual cells and nanolitre reaction volume. Typically, full-length cDNA libraries generated from these platforms undergo fragmentation and PCR enrichment of the 3′ end of molecules containing the cell barcode so they are suitable for short-read sequencing. In contrast to existing methods, the inventors designed a strategy which involved splitting full-length single-cell 3′-tag or 5′-tag cDNA libraries prior to fragmentation for short-read sequencing, and selectively enriching BCR and TCR transcripts using targeted hybridization capture. Targeted capture was chosen over commonly used PCR methods (Carlson et al., (2013) Nature communications, 4:2680; Shugay et al., (2014) Nature methods, 11(6):653-5) to retain full-length transcripts. Enriched antigen-receptor molecules were then subjected to long-read Nanopore sequencing to obtain both the 3′ or 5′ cell-barcode and the 5′ VDJ sequence. In parallel, short-read Illumina sequencing was performed to profile gene expression on the remaining cDNA. By matching the cell barcodes obtained from long-read sequencing with the cell barcodes obtained from Illumina sequencing, transcriptome profiles of individual cells could be linked with clonotype sequence (FIG. 1).

The inventors also designed a capture bait library with biotinylated probes specifically targeting all annotated and functional human V, J and Constant (C) region exons within the genomic loci that encode TCRα, TCRβ, TCRd, TCRg, IgH, Igκ and Igλ chains. Capture probes were chosen over V-region specific PCR primers to minimize preferential bias introduced during multiplex PCR (Carlson et al., 2013) and to retain full-length transcripts. The capture bait library also included probes specific for the PTPRC transcript, encoding the CD45 protein. Activation of lymphocytes by antigen causes a switch in PTPRC splicing, resulting in the expression of unique CD45 proteins on the cell surface. These can be used to distinguish naive from antigen-experienced lymphocytes and so are very informative in the study of lymphocyte biology. However, PTPRC splice variants cannot be accurately measured using short-read sequencing.

Oxford Nanopore Technologies (ONT) sequencing was chosen to read full-length cDNA molecules due to its high-throughput and low cost. The major challenge of generating highly accurate clonotype sequences with long-read technologies, however, is the high error rate, estimated at ˜10% error per nucleotide. Whole genome assemblies generated from long-read sequencing often overcome this error limitation through the use of de novo assembly followed by ‘polishing’ to achieve high accuracy of over 99% (Jain M., et al. (2018) Nature Biotech). It was predicted that such approaches could also be applied to Nanopore reads generated from cDNA targeted capture and a computational pipeline was developed that performs de novo assembly on demultiplexed Nanopore data to generate full-length clonotype sequences for each cell (FIG. 2).

The RAGE-seq pipeline is more fully illustrated in the flowchart presented in FIG. 1, and the general methodologies are described in further detail below.

### 1.2 GENERAL METHODOLOGIES

### 1.2.1 Patient Samples

Patient tissues used in this work were collected under protocol X13-0133, HREC/13/RPAH/187. HREC approval was obtained through the SLHD (Sydney Local Health District) Ethics Committee (Royal Prince Alfred Hospital zone), and site-specific approvals were obtained for all additional sites. Written consent was obtained from all patients prior to collection of tissue and clinical data stored in a de-identified manner, following pre-approved protocols. Tissue analysis was performed under protocol x14-021, LNR/14/RPAH/155.

### 1.2.2 Single-Cell Suspension Preparation

Following surgical resection of tumour and lymph node from patient, samples were transferred in ice cold RPMI-1640 with 50% FCS to the laboratory to be processed. Tumour was cut into approximately 1 mm³ pieces and dissociated as per MACS human tumour dissociation kit (Miltenyi Biotec, Australia). Lymph node was similarly processed however with digestion halted at 15 minutes. After washing twice with 2% FBS in PBS, cells were resuspended in sorting buffer and passed through 70 um strainers. The Jurkat T-cell line and Ramos B-cell line were cultured in RPMI-1640 medium with 10% FCS. Monocytes were flow sorted from human peripheral blood mononuclear cells (PBMCs) using a human anti-CD14 antibody. Flow cytometric sorter (BD FACS AriaIII) was used enrich for viable cells using DAPI stain, maintaining a gating threshold which omits red blood cells. Cells were centrifuged and resuspended in PBS with 2% FCS to obtain an approximate concentration of 1000 cells/μ1 which was counted using a haemocytometer. Samples were always handled on ice when possible and a viability of at least ˜90% for all samples was confirmed by trypan blue stain prior to capture.

1.2.3 Droplet Based scRNAseq (10× Genomics) Capture was performed as per 10× Chromium Single Cell 3′ (V2 chemistry) protocol, aiming for an estimate of 4000 captured cells for each sample. Full-length cDNA was split 1:1 for Nanopore long-read sequencing and for short read sequencing. An Illumina NextSeq 500 was used to sequence the transcriptome library, and the yielded raw bcl file was demultiplexed and aligned (hg38 build) using CellRanger 2.0 (10× Genomics).

### 1.2.4 Antigen-Receptor Capture Probe Design

A target enrichment library (Roche NimbleGen) was designed by first identifying gene annotations of all functional V (IGHV, IGKV, IGLV, TRAY, TRBV, TRGV), J (IGHJ, IGKJ, IGLJ, TRAJ, TRBJ, TRGJ, TRDJ), and C (IGHA, IGHD, IGHE, IGHG, IGHM, IGKC, IGLC, TRAC, TRBC, TRGC, TRDC) and PTPRC genes obtained from the IMGT database [doi: 10.1093/nar/gku1056]. For each gene, genome coordinates of their corresponding exons were obtained from the GRCh38 primary assembly. Design of probes from target regions and synthesis was performed by Roche NimbleGen using the SeqCap RNA Choice format with a maximum of 5 matches to the human genome. 66 regions were removed from the final design due to being too small according to the NimbleDesign tool. In total 678 exons were targeted by the CaptureSeq array targeting ˜128 Kb.

### 1.2.5 Targeted Capture

Following full-length cDNA amplification one tenth to one half of the total volume of cDNA was used for targeted capture and nanopore sequencing. Pre-capture PCR was first performed using KAPA-Hi-Fi polymerase, 3 mM TSO and 3 mM R1 primer with the following cycling conditions: 98° C. for 3 min; 98° C. for 20 s, 65° C. for 30 s, 72° C. for 1 min 30 s×5 cycles (cell lines) or ×20 cycles (primary cells). Next PCR products were purified using AMPure XP beads and 500 ng-1 ug of amplified cDNA was used for targeted capture using the protocol previously described (Mercer et al., (2014) Nature protocols, 9(5):989-1009), with the following modifications. Universal hybridisation enhancing (HE) oligo and index HE oligos were not included during hybridisation. Two rounds of hybridisation were performed for 24 h each at 47° C. Following each round of hybridisation and capture, PCR was performed using KAPA hi fi instead of Phusion DNA polymerase with 1 mM TSO primer and 1 mM R1 primer (instead of the TS-PCR oligos) with the following PCR cycling conditions: 98° C. for 3 min; 98° C. for 20 s, 65° C. for 15 s, 72° C. for 1 min 30 s×5 cycles (first round) or ×20 cycles (second round). Postcapture cDNA library size ranged from 0.6 to 2 kb.

### 1.2.6 SmartSeq2

SmartSeq2 was performed as described by Picelli et al., (2014) Nature protocols, 9(1):171-81 with the following modifications: the IS PCR primer was reduced to a 50 nM final concentration and the number of PCR cycles increased to 28. Sequencing was performed on the Illumina NextSeq platform.

1.2.7 scRNAseq Count Matrix Processing

The raw gene expression matrices were normalised and scaled using Seurat (v3.4) (Satija et al., (2015) Nature biotechnology, 33(5):495). For the cell line capture, cells that express <250 genes or <1000 UMIs or that contain more than 6% UMIs derived from mitochondrial genome were excluded. To reduce doublet contamination, any cells expressing >6500 genes were discarded, additionally removing any cells that are ×5 deviated from the median gene count for that cell type. For lymph node and tumour, a threshold of <100 genes or <500 UMIs was set to allow detection of exhausted T-cells. A principle component analysis was performed on the variable genes and by using the Jackstraw method, the first principle components with a P-value<0.01 was used for dimensional reduction. For Tumour and Lymphnode combined analysis, Seurat's RunCCA was used for cross-dataset normalization to enable subsequent comparative analyses. Cell cycle scoring was performed using scRNA cell cycle gene expression scores from Nestorowa et al., (2016) Blood, 128(8): e20-31 and Tirosh et al., (2016) Science, 352(6282):189-96.

### 1.2.8 Identification of Major Clusters

The resolution set for each tSNE analysis was determinant on the strength of annotation using well known canonical marker genes and Seurat's FindAllMarkers function yielding an average expression for any particular cluster which yielded >2.5-fold higher than the average expression in other sub clusters from that cell type.

### 1.2.9 Nanopore Sequencing

Hybridisation capture cDNA libraries were prepared for long read sequencing using Oxford Nanopore Technologies' (ONT) 1D adapter ligation sequencing kit (SQK-LSK108), with the exception of one sample that used the 1D² adapter ligation kit (LSK-308). The latter was base called and considered as 1D for all subsequent steps. All samples were sequenced with R9.4.1 flowcells (FLO-MIN106), with the exception of 3/6 cell line samples that were loaded onto R9.5.1 (FLO-MIN107) flowcells (including the aforementioned LSK308 sample). Base calling was performed offline on a high-performance computing cluster using ONT's Albacore software pipeline (version 2.2.7). A list of samples, chemistries, flowcell identification numbers, and manufacturer software versions can be found in Table 1.

TABLE 1
List of samples, chemistries, flowcell identification
numbers, and manufacturer software versions.

| | | | | Albacore |
|---|---|---|---|---|
| Sample | Flowcell ID | Flowcell chemistry | Kit | version |
| Tumour | FAH59175 | FLO-MIN106 | SQK-LSK108 | 2.2.7 |
| | FAH63491 | | | |
| | FAH77585 | | | |
| | FAH77358 | | | |
| | FAH53149 | | | 2.1.3 |
| | FAH89946 | | | Guppy |
| Lymph Node | FAH59253 | FLO-MIN106 | SQK-LSK108 | 2.2.7 |
| | FAH60789 | | | |
| | FAH77356 | | | |
| | FAH82913 | | | |
| | FAH52286 | | | 2.1.3 |
| | FAH86252 | | | NA |
| | FAH82560 | | | |
| Ramos & | FAH58575/FAH58565 | FLO-MIN106 | SQK-LSK108 | 2.2.7 |
| Jurkat | FAH60746 | | | |
| | FAH84424 | | | |
| | FAH04597 | FLO-MIN107 | LSK308 (basecalled SQK-LSK108) | 2.1.3 |
| | FAH04593 | | SQK-LSK108 | |
| | FAH30303 | | | |

### 1.2.10 Demultiplexing Nanopore Sequencing Data

Base called fastq files were pooled for each biological sample and subjected to ad hoc demultiplexing using a direct sequence matching strategy (i.e. 0 mismatches and indels). Cell barcode sequences (16 nt) were extracted from matched short read sequencing data, as produced by 10× Genomic's CellRanger software. Forward and reverse-complemented cell barcode sequences were then used to demultiplex the nanopore sequencing reads. This was achieved by scanning the first and last 200 nt of any read longer than 250 nt for an exact match to the list of barcodes (10× genomics). The reads were trimmed by ‘chopping’ the read 13 nt downstream of the position matching a cell barcode to ensure that (i) the 10 nt UMI sequence is removed from consensus assembly steps, and (ii) potential insertions are also removed, which may also remove a few bases of the poly-T/Ail. The fastq headers were modified to include barcode and UMI sequences post-demultiplexing.

### 1.2.11 De Novo Assembly and Error Correction

As highlighted in FIG. 1 and FIG. 2, demultiplexed long-sequencing reads were grouped into distinct fastq files and subjected to de novo assembly using Canu (Koren et al., (2017) Genome Research, 27:1-15) to generate contigs, then the long sequencing reads were re-aligned to the resulting contigs with minimap2 (Li H. (2018) Bioinformatics, 34(18):3094-3100). In addition to de novo assembly, targeted or ‘baited’ assembly was also performed, where, instead of generating contigs, the complete nucleotide sequence of all targets (or ‘baits’) used to design probes for targeted capture (c.f. Method section 1.2.4) was used as a reference against which long sequencing were first aligned to using minimap2. This effectively results in an in silico capture to reduce assembly artifacts. Contigs or ‘baited’ alignments were corrected (for errors) with Racon (Vaser R et al., (2017) Genome Research) and ‘polished’ with raw nanopore sequencing data using nanopolish (https://github.com/jts/nanopolish) to produce consensus sequences. These steps were run on a Sun Grid Engine high-performance computing cluster and are summarised in (FIG. 2b).

### 1.2.12 TCR and BCR Clonotype Assignment

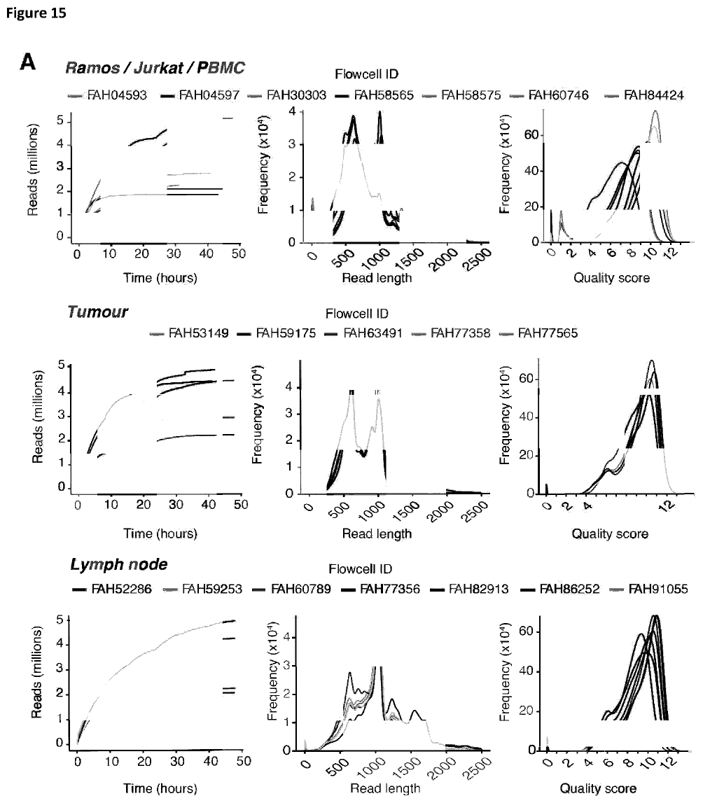

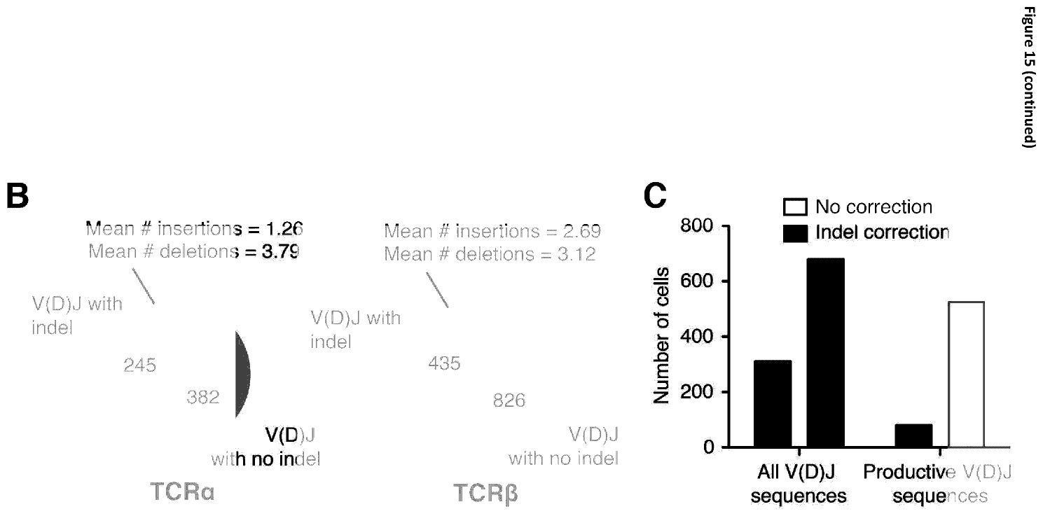

Polished fasta files containing consensus transcript contigs for each cell barcode were subjected to IgBLAST (Ye et al., (2013) Nucleic acids research, 41(Web Server issue):W34-40) alignment to determine V(D)J rearrangements and blastn alignment (Camacho et al., (2009) BMC bioinformatics, 10:421) to determine the Ig or TCR constant regions exons associated with the V(D)J. For each contig, separate IgBLAST for immunoglobulin and TCR were performed using IMGT germline gene reference datasets (Lefranc et al., (2015) Nucleic acids research, 43(Database issue):D413-22). Amino acid sequences and location of CDR3 were defined by the conserved cysteine-104 and typtophan-118 based on the IMGT numbering system (Lefranc et al., (2015) Nucleic acids research, 43(Database issue):D413-22). IgBLAST parameters were default with the exception of returning only a single gene segment per V(D)J loci. Text-based IgBLAST output was then parsed to tab-delimited summaries, calling gene segments, framework and complementarity determining regions, mismatches, and indels relative to germline gene segments. Following this first round of IgBLAST, insertions and deletions (indels) in parts of the sequence that aligned to germline gene segments were corrected to their closest germline gene, and the IgBLAST step was repeated to generate indel corrected alignments. This was particularly important for correction of Nanopore sequencing errors that would otherwise impact on the reading frame of the V(D)J rearrangement that would prevent the CDR3 from being determined accurately. The impact of correcting insertions/deletions (indels) is shown in FIG. 15.

### 1.2.13 Clonotype Filtering

Clonotypes that were out-of-frame or that contained stop codons, termed non-productive clonotypes, were removed unless stated otherwise. BCR clonotypes containing more than 40 mutations or TCRs with more than 5 mutations in their respective V gene segments were filtered against. Analysis of SHM of Jurkat V regions included no filtering of clonotypes based on number of mutations (FIG. 3). If a cell was assigned two different TCR clonotypes with the same V and J genes but different CDR3 amino acid sequences, the TCR clonotype with the greater number of mutations in the V region was removed. If there were no differences in V region mutations the clonotype with the lowest number of reads used in assembly was removed. For BCR clonotypes only assembly read coverage was used for filtering.

### 1.2.14 CD45 Isoform Assignment

Several methods for isoform assignment can be employed from the alignments of either raw reads or consensus transcript contigs to the canonical CD45 splice variants. For this example, depth of coverage and coordinates of the raw reads for each cell barcode that span exon 1-7 can be used to assign likelihood of belonging to a particular CD45 isoform. CITE-Seq data can be used to validate isoform assignment.

### 1.2.15 CD45 Isoform Filtering

Of the cells that aligned to canonical CD45 splice variants, cells that were missing either exon 3 or exon 7 of CD45 were removed (all known CD45 isoforms should include these exons). Cells with less than 50 reads were removed.

### 1.2.16 Assignment of Splice Isoforms

To determine the spliced constant regions exons that were associated with the V(D)J rearrangement blastn was used to align each contig against the spliced reference exons. For the IGHC, both the membrane and secreted versions of each constant region were included. Tabular blastn output was parsed to call constant region for each contig using the criteria of greater than 95% coverage of the spliced constant region exons and percentage identity of more than 90%. A 90% identity threshold was used as contigs used for constant region calling were not indel corrected.

1.2.17 Integration of Clonotype with scRNA-Seq

Clonotypes that define groups of cells that are likely to have arisen from clonal expansion of the same progenitor B or T cell were defined either be shared gene rearrangements using the same V and J germline gene segments with identical CDR3 amino acid sequences for the T cells, and same V and J germline gene segments with 90% identical CDR3 nucleotide sequence for B cells. Clonotypes either shared the same paired chains (e.g., heavy and light chains for BCR, and alpha/beta or gamma/delta chains for the TCR) or shared TCRβ or IGH chains.

### 1.2.18 Read Subsampling

Read subsampling was performed on 200 Jurkat and 200 Ramos cells with each cell having no less than one thousand reads. The subsampling itself was performed with the sequence analysis toolkit, seqtk version 1.0-r72 (https://github.com/lh3/seqtk), using the sample command with a seed parameter of −s123. Subsampling was performed in a stepwise manner at increments of 1000, 500, 250, 100 and 50 read depths, with the resulting subsampled fastq the next input in later rounds of subsampling.

### 1.2.19 Determining On-Target Nanopore Alignments

Alignment of nanopore reads to TCR and BCR genes was performed by the alignment program Minimap2 version 2.3-r536 (Li H. (2009) Bioinformatics (Oxford, England)) to a custom reference fasta sequence containing TCR and BCR constant region genes, using the ‘-x map-ont’ preset. The resulting alignments were sorted and then viewed using samtools (Li et al., (2009) Bioinformatics (Oxford, England), 25(16):2078-9) version 1.7-2-gc6125d0 (with htslib 1.7-6-g6d2bfb7) and reads flagged as unmapped, not primary or supplementary were not counted as on-target.

### 1.2.20 On-Target Nanopore CD45 Alignments

Nanopore reads were aligned to the CD45 primary assembly (GRCh38.p12) as previously described, then aligned to a custom reference fasta sequence containing CD45 exon sequences that discriminate into canonical CD45 splice variants (i.e. RABC, RO, etc.). Depth of coverage across these splice variant sites was examined using Samtools version 1.7-2-gc6125d0 (with htslib 1.7-6-g6d2bfb7) via the depth command.

### 1.2.21 Determination of Cell Surface Protein Marker Expression Using CITE-Seq

CITE-Seq is a method to permit simultaneous measurement of transcriptomes and cell surface epitopes using barcoded antibodies. (doi:10.1038/nMeth.4380). Essentially as described, cell suspensions were stained with a pool of 87 uniquely barcoded antibodies (purchased from Biolegend Inc) prior to capture using the 10× genomics system. Following size fractionation of cDNAs, CITE-Seq libraries were sequenced separately using an Illumina Next-Seq platform and analysed using Seurat.

### 1.3. CROSS-PLATFORM SEQUENCING VALIDATION

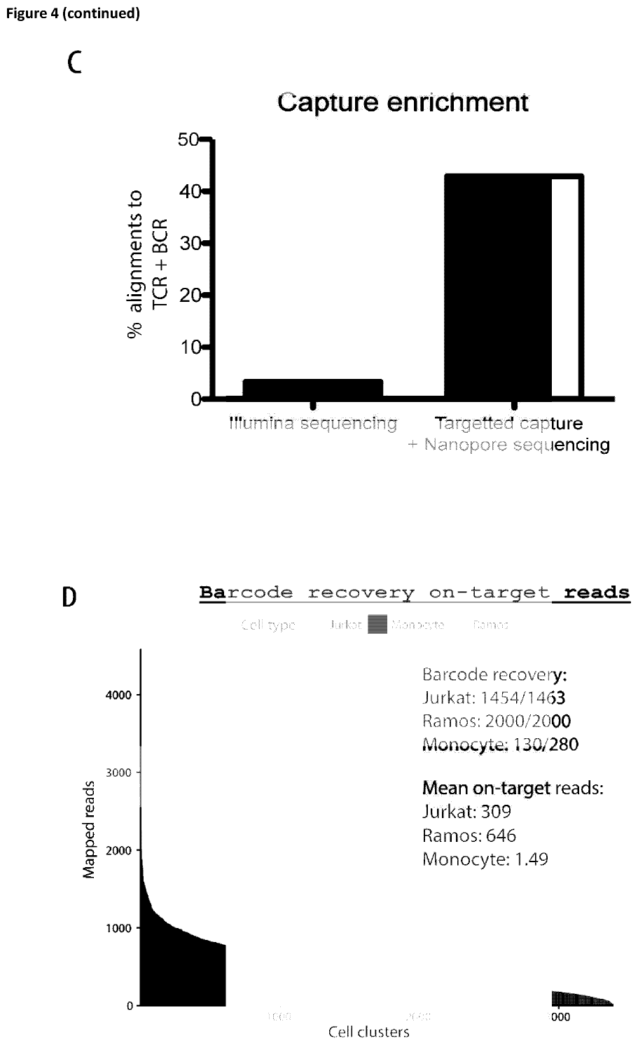

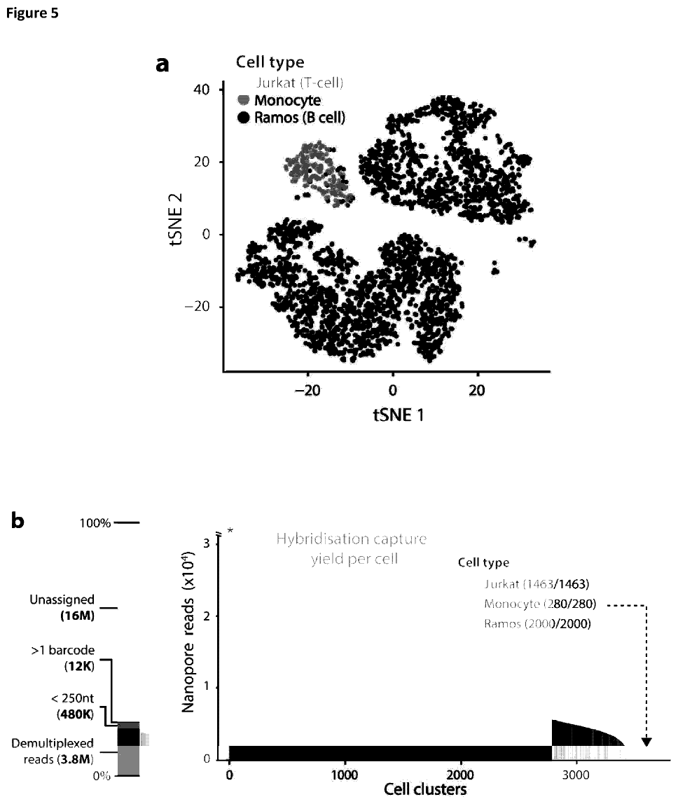

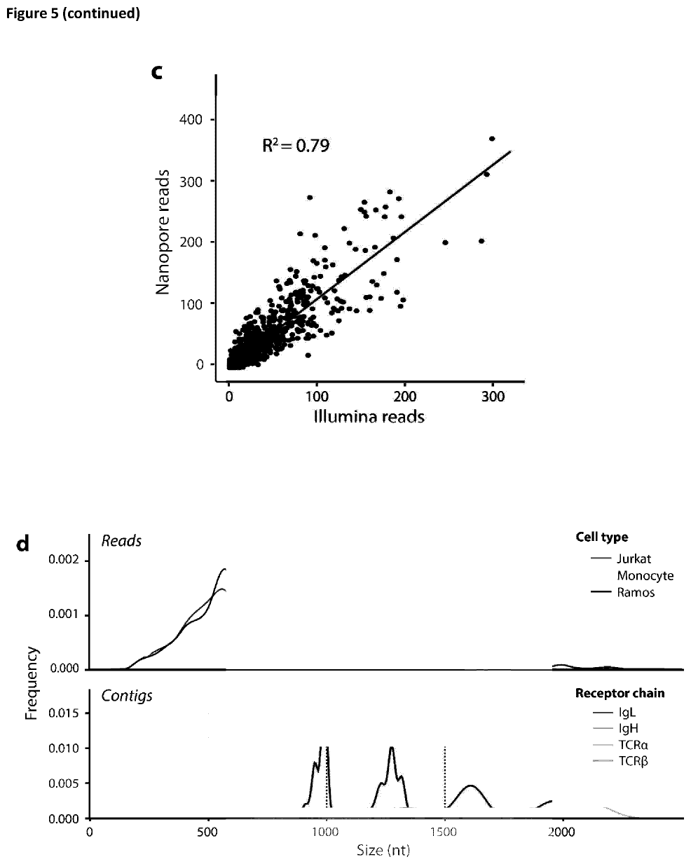

To assess the validity of this method, the inventors then performed RAGE-Seq on a mixture of the human T cell line Jurkat and the human B cell line Ramos, for which antigen receptor sequences are published (FIG. 4). A proportion of ˜15% human monocyte cells were added to serve as a negative control. This mixture of cells was partitioned using the 3′ 10× Chromium platform (Zheng et al., (2017) Nature communications, 8:14049) and following full-length cDNA amplification, the cDNA was split for both standard 10× protocol and Illumina sequencing, and targeted enrichment followed by Nanopore sequencing. The dataset consisted of 1463 Jurkat cells, 2000 Ramos cells and 280 monocytes (FIG. 5A; FIG. 4). Following nanopore sequencing, a total of 20,346,396 nanopore reads were obtained, 42.9% of which uniquely aligned to TCR and BCR constant regions (on-target reads), representing an ˜13-fold enrichment when compared to non-targeted capture Illumina data (FIG. 4).

To demultiplex 10× cellular barcodes from the nanopore sequencing reads, a whitelist of cell barcodes from Illumina sequencing was generated and used to search for a direct match within each read. Using this approach, 3,805,076 de-multiplexed reads (18.7%) containing all of the 10× cell barcodes (FIG. 5B) was recovered. This demonstrates that the amplified full-length cDNA library can be sufficiently sampled between the two platforms. Barcode recovery for Nanopore reads that were on-target was 99.3% and 100% for Jurkat and Ramos cells, respectively, and 46.4% for monocyte cells (FIG. 4). A strong correlation between Nanopore and Illumina reads was also observed (FIG. 5C).

### 1.4 IDENTIFICATION OF RECEPTOR CLONOTYPES

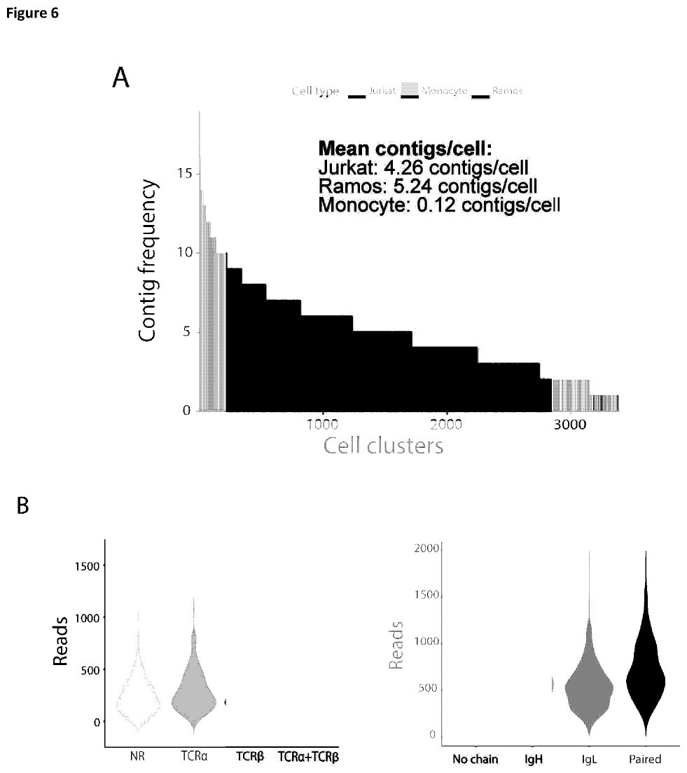

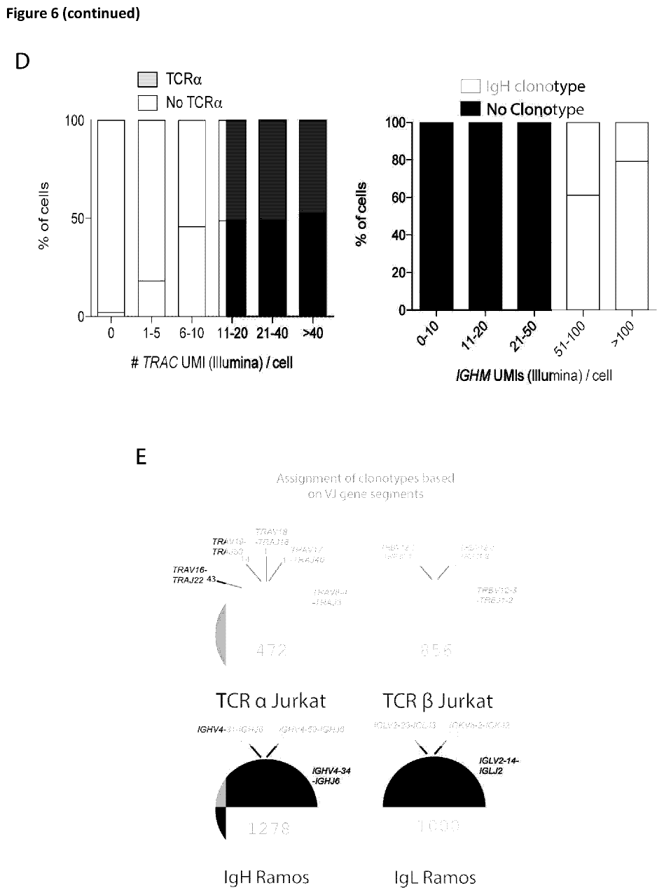

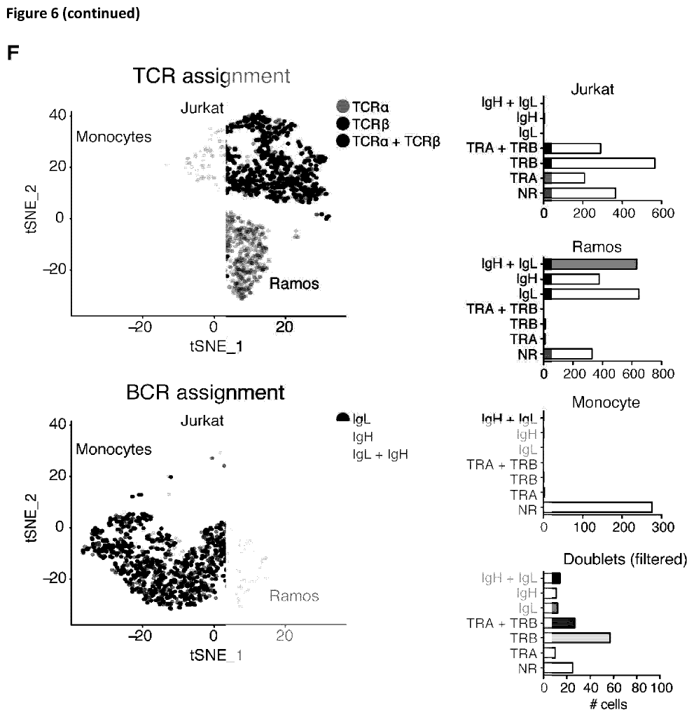

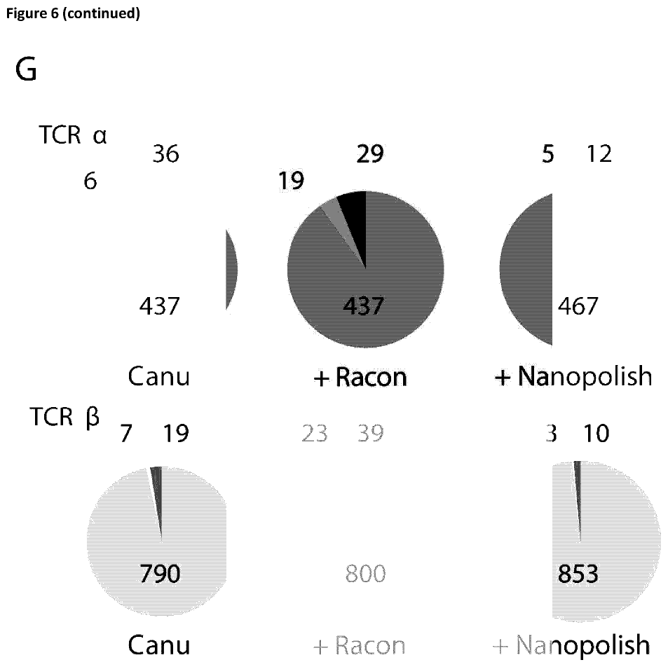

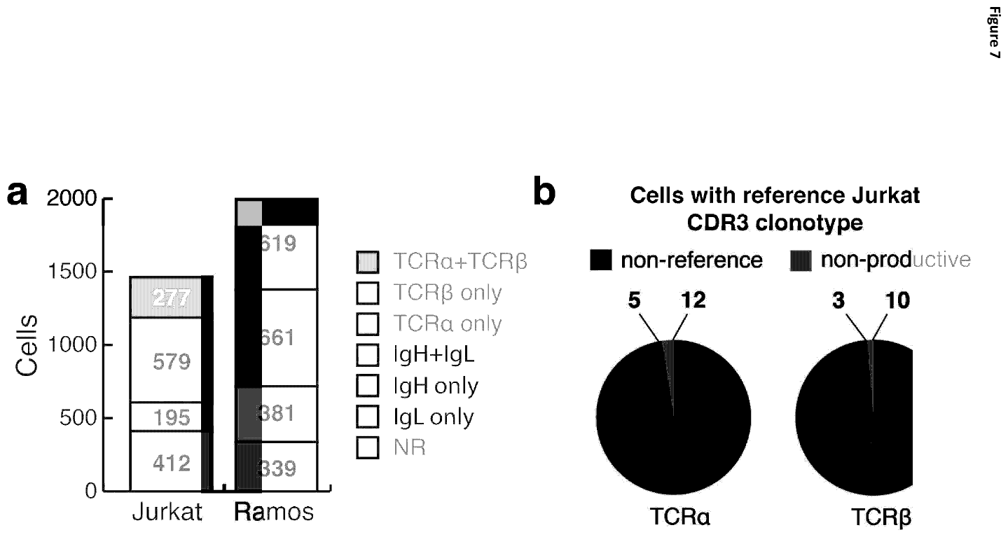

The inventors also carried out de novo assembly, error correction and contig polishing on the nanopore reads (see Methods), generating on average 4.26 contigs per Jurkat cell, 5.24 per Ramos cell and 0.12 per Monocyte (FIG. 6). On average, 30% of contigs for Jurkat cells and 32.9% of Ramos cells were assigned a productive clonotype. The nucleotide length of the recovered clonotype sequences were found to overlap with the nanopore sequencing read length over 0.9 kb and were consistent with the predicted full-length Jurkat TCRα and TCRβ and Ramos IgH and IgL reference mRNA transcripts (FIG. 5D; FIG. 6). Importantly, this shows that the de novo assembly approach can retain full-length transcripts.

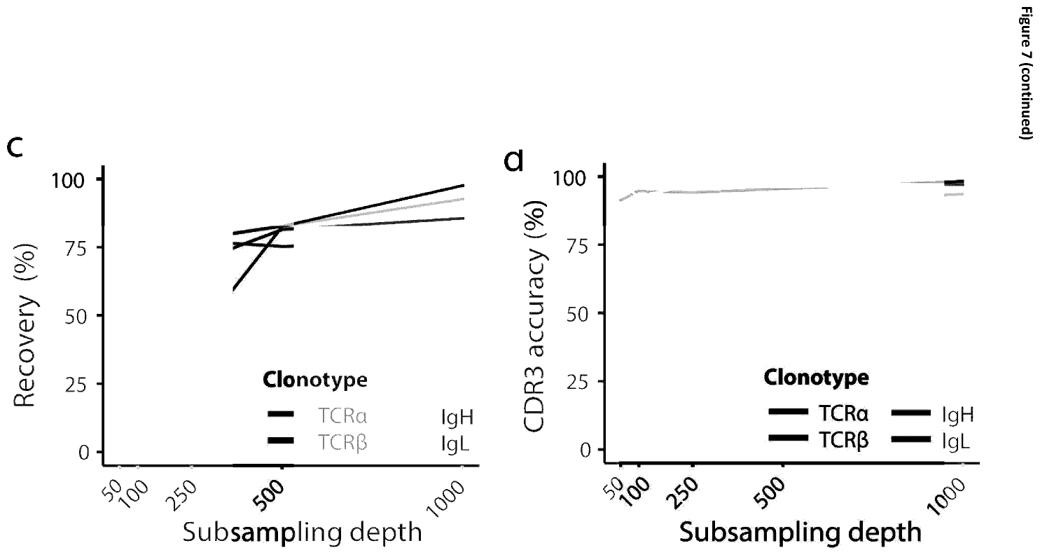

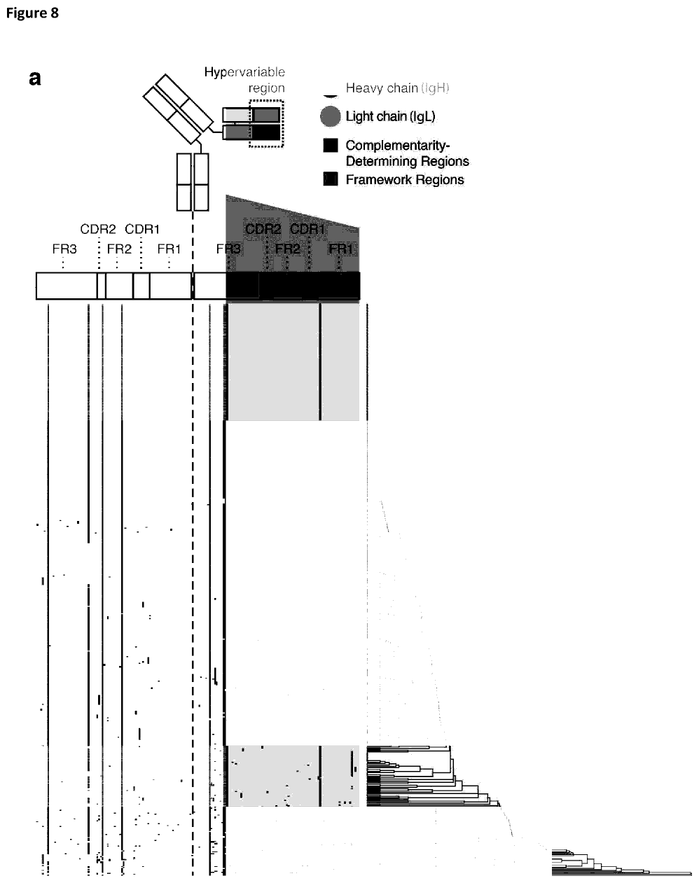

For Jurkat cells, paired TCRα and TCRβ chains were recovered from 18.9% of cells, 13.3% with a TCRα chain only and 39.6% with a TCRβ chain only (FIG. 7A). For Ramos, paired IgH and IgL chains were recovered from 31% of cells, 33% with a IgH chain only and 15.9% with a IgL chain only (FIG. 7A). There was little assignment of non-reference VJ-gene pairs (FIG. 6E).

Next, the accuracy of calling a correct clonotype at nucleotide resolution was evaluated by investigating the CDR3 region of Jurkat cells against their known reference CDR3 sequences (FIG. 6). The percentage of Jurkat cells expressing the reference CDR3 was very high. 98.9% expressed the reference CDR3a sequence and 99.6% (853/856) expressed the reference CDR3b sequence while the number of cells carrying non-productive sequences was small (FIG. 7B). Assembly polishing was found to modestly increase the recovery of cells with productive clonotypes (3.15% for TCRα and 6.14% for TCRβ) and had a small effect on the overall accuracy (FIG. 6G). It was also found that read depth impacted the total number of clonotypes recovered, but had little effect on the CDR3 accuracy for both TCRα and TCRβ (FIGS. 7C and 7D). When performing targeted (or ‘baited’) assembly—where long reads were first aligned to the sequences corresponding to the target genes or regions—less reads are required to recover accurate clonotypes (FIG. 14).

RAGE-Seq was also compared against the reconstruction of Jurkat TCR sequences produced using SmartSeq2 using VDJ-Puzzle (Eltahla et al., (2016) Immunology and cell biology, 94(6):604-11). VDJ-Puzzle was able to recover more TCR clonotypes at high accuracy. However, RAGE-Seq proved to be ˜30 times more cost effective on a per cell basis (Table 2). Taken together, these results indicate that RAGE-Seq is both accurate and sensitive in determining clonotype sequences and has significant advantages over SmartSeq2 in terms of cost and throughput.

TABLE 2
Number of total Nanopore reads and de-multiplexed Nanopore
reads per sample.

| Sample | Total reads | Total on-target reads | Percent de-multiplexed |
|---|---|---|---|
| Cell line | 20,346,396 | 3,805,076 | 18.7 |
| Lymph node | 21,721,568 | 3,380,621 | 19.9 |
| Tumour | 16,601,436 | 3,069,468 | 15.8 |

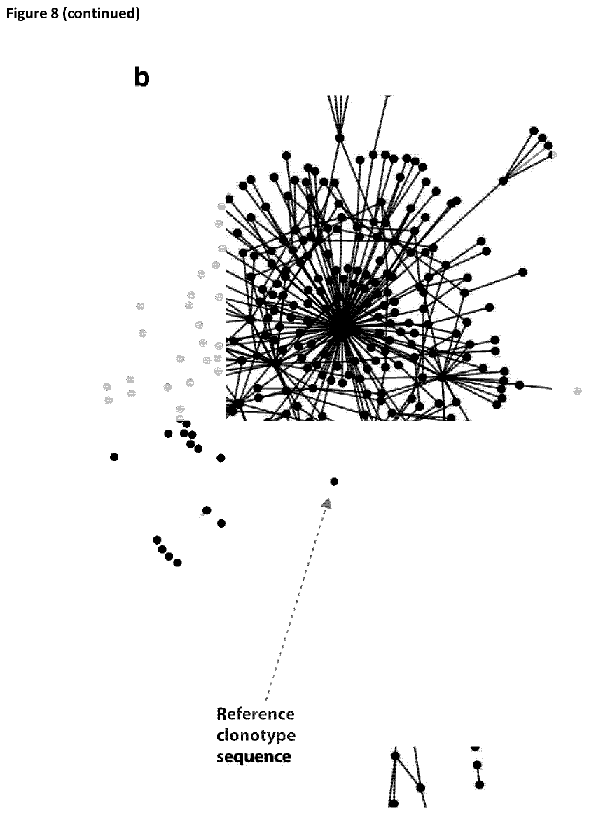

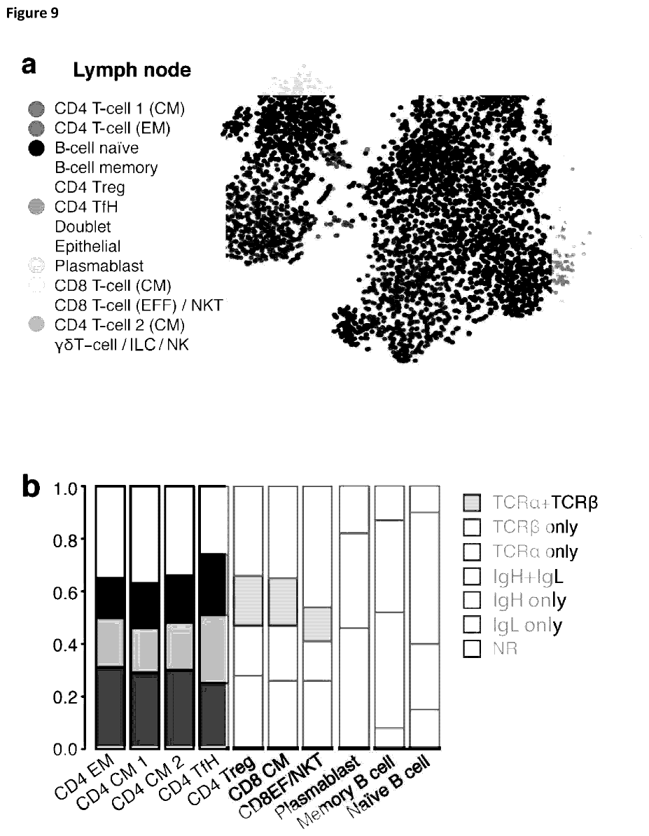

B cells can acquire additional BCR diversity through somatic hypermutation (SHM) of variable regions of immunoglobulin genes. The Ramos cell line is known to mutate its receptors by undergoing SHM in culture (Sale and Neuberger (1998) Immunity, 9(6):859-69). To assess the impact of SHM on BCR diversity, accurate sequence across the entire V region of the heavy and light chain is required. Here, RAGE-Seq was able to recover over 99% of Ramos IgH and IgL clonotypes with the complete V region length (FIG. 3). Amino acid replacement mutations were determined across the entire V regions of IGHV and IGLV from 615 Ramos cells assigned paired IgH and IgL receptors. Conserved amino acid mutations were observed in six different IgH and IgL positions, including mutation within the hydrophobic patch in FR1 of IgH (residues 23-25) which promotes self-reactivity (Potter et al., (2002) Journal of immunology (Baltimore, Md.: 1950), 169(7):3777-82) (FIG. 8A). A dominant subclone within the Ramos cell line was represented by 147 cells along with 37 subclones represented by more than one cell and 319 subclones represented by a single cell (FIG. 8A). A clone network was generated based on nearest neighbour distance that included the inferred germline sequence as the unmutated ancestor (FIG. 4B), demonstrating the evolution of individual Ramos cells undergoing active somatic hypermutation. Thus, RAGE-seq can pair transcriptomic phenotype to immunoglobulin sequences of individual clone members within clonal populations.

Jurkat TRAV (TCRα) and TRBV (TCRβ) genes were then interrogated to assess the accuracy of RAGE-seq to call SHM, which should be completely conserved in this clonal cell line. A low number of Jurkat cells with one or more nucleotide mismatches to germline in these regions were identified (TRAV: 5.05%, TRBV: 2.8%, FIG. 3). Additionally, read subsampling of both Jurkat and Ramos cells found little effect of read coverage on calling SHM events (FIG. 3).

### 1.5. ANALYSIS OF LYMPHOCYTES FROM A HUMAN LYMPH NODE

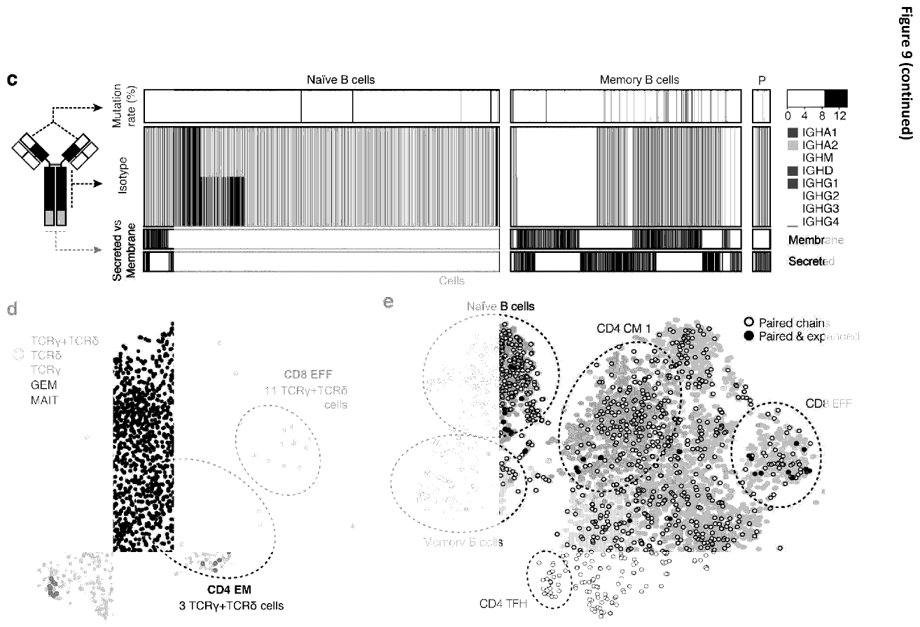

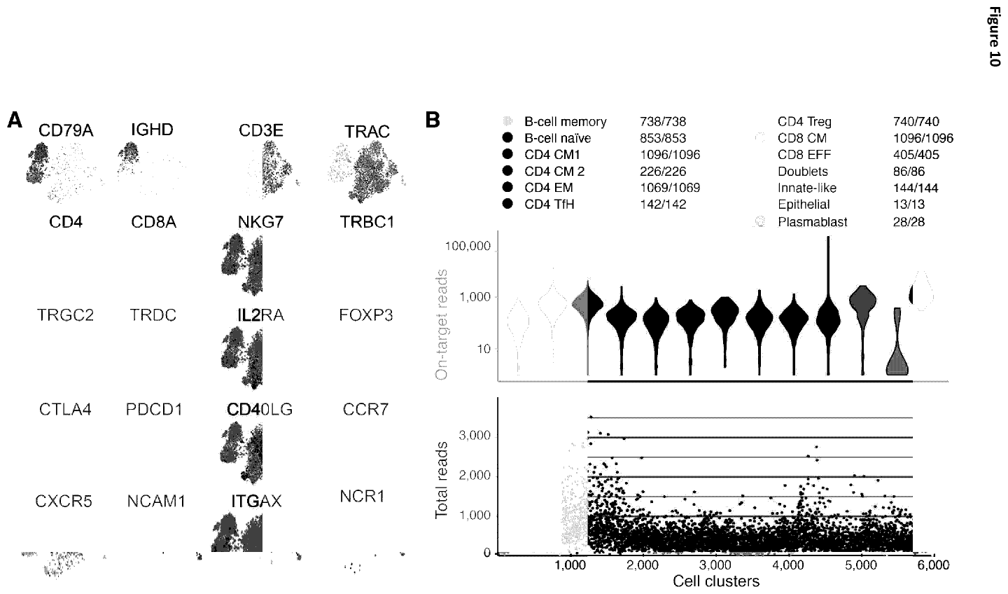

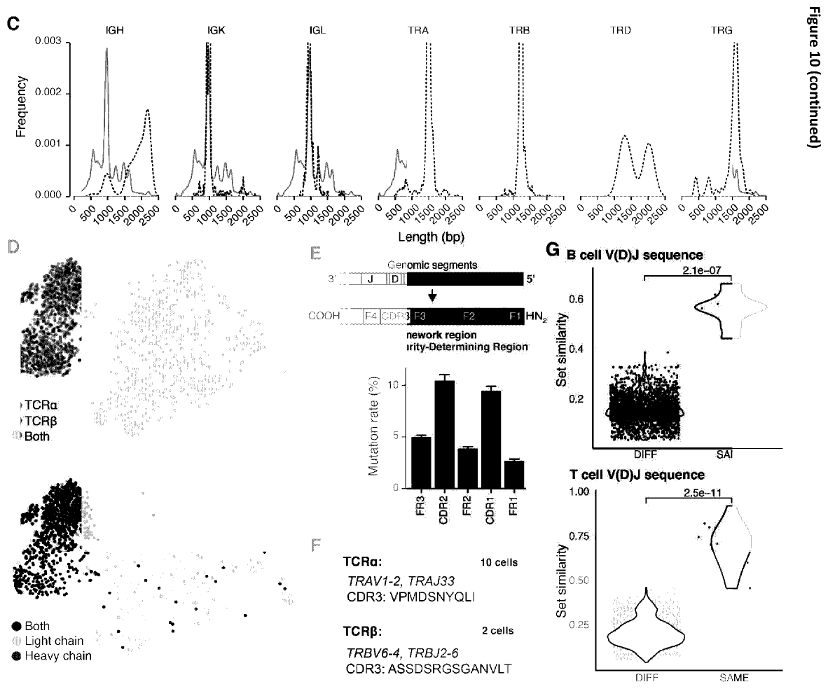

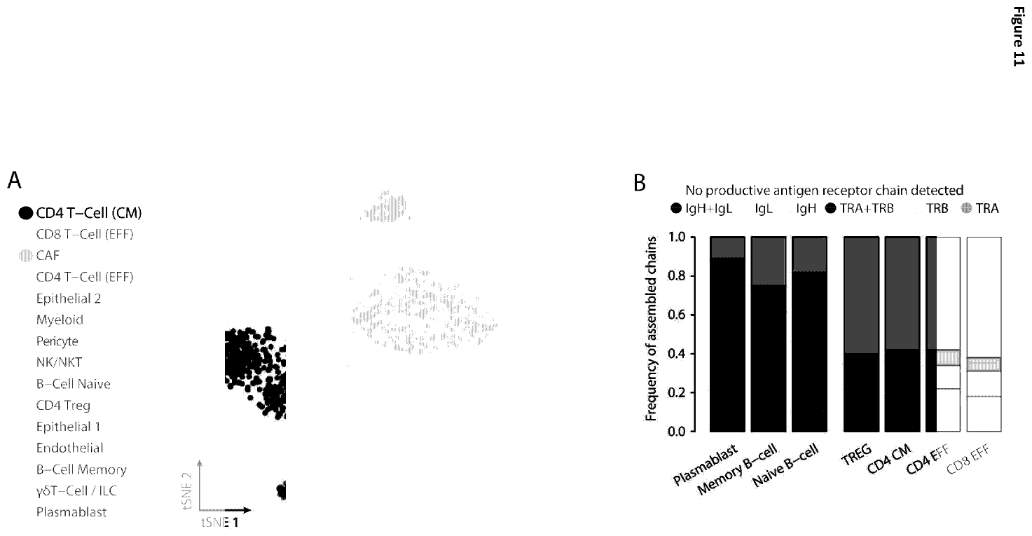

RAGE-Seq was then performed on a human lymph node resected from a breast cancer patient in order apply the method to primary B and T lymphocytes. In doing so, 4,165 T cells were identified which could be subdivided into 6 clusters: CD4 effector memory (EM; 1069 cells), CD4 central memory (CM; 1321 cells), CD4 T follicular cells (TfH; 142 cells), CD4 T regulatory cells (Treg; 740 cells), CD8 CM (487) and CD8 effector (EF)/NKT (405 cells) (FIG. 9, FIG. 10) Among all primary T cells, 705 (16.9%) cells were recovered with paired TCRαβ chains, 1199 (28.7%) cells with a TCRα chain only and 762 (18.3%) cells with a TCRβ chain only. The recovery rate of TCR chains was comparable across the different T cell subsets (FIG. 9B). It was also possible to detect two different TCRα or TCRβ clonotypes in 138 (9.5%) and 35 (1.8%) T cells respectively, a frequency similar to previous reports (Stubbington et al., (2016) Nature methods, 13(4):329-32; Eltahla et al., (2016) Immunology and cell biology, 94(6):604-11). Among the 1619 B cells in the lymph node, 689 cells were recovered with paired IgH and IgL chains, 188 cells with only a IgH chain and 557 cells with only a IgL chain (FIG. 5B). Similar to the cell line experiment, all the cell barcodes were recovered across both sequencing platforms and full-length clonotype sequences were assembled (FIG. 10).

The targeted capture panel included probes against TCRγ and TCR allowing for the detection of TCRγδ cells, a poorly-explored class of unconventional T cell, of substantial interest to studies of infection and tumour immunology. A total of 11 T cells in the lymph node were assigned paired TCRγδ chains, the majority of which clustered in the CD8 EFF cluster. 92 T-cells were recovered with only the TCRγ chain and 14 T cells with only the TCR chain only, again the majority of which clustered in the CD8 EFF population (FIG. 5C). T cells assigned TCRγ chains alone were found to frequently co-express TCRα and TCRβ chains, consistent with the timing of TCRg rearrangement (Joachims et al., (2006) Journal of immunology (Baltimore, Md.: 1950), 176(3):1543-52). In contrast, T cells assigned paired TCRgd chains did not co-express TCRα or TCRβ clonotypes suggesting that they are true TCRγδ cells. The inventors also explored the identification of other unconventional T cells that can recognise non-peptide antigens based on their invariant TCR usage such as Mucosal Associated Invariant T (MAIT) cells and Germline-Encoded Mycolyl lipid-reactive (GEM) T cells. 10 T cells were found to carry MAIT-associated TCRs which clustered closely together in the CD8 effector population, while two T cells with GEM-associated TCR chains were found in the CD4 effector memory cluster (FIG. 9D). Interestingly the T cells carrying MAIT-associated TCRs all comprised of a single expanded clone (FIG. 10F).

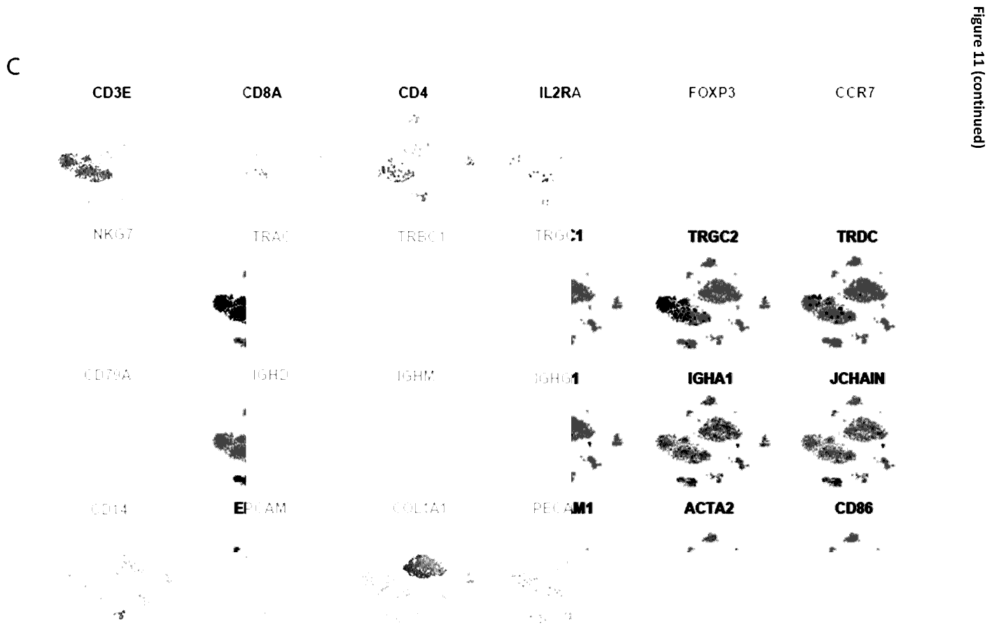

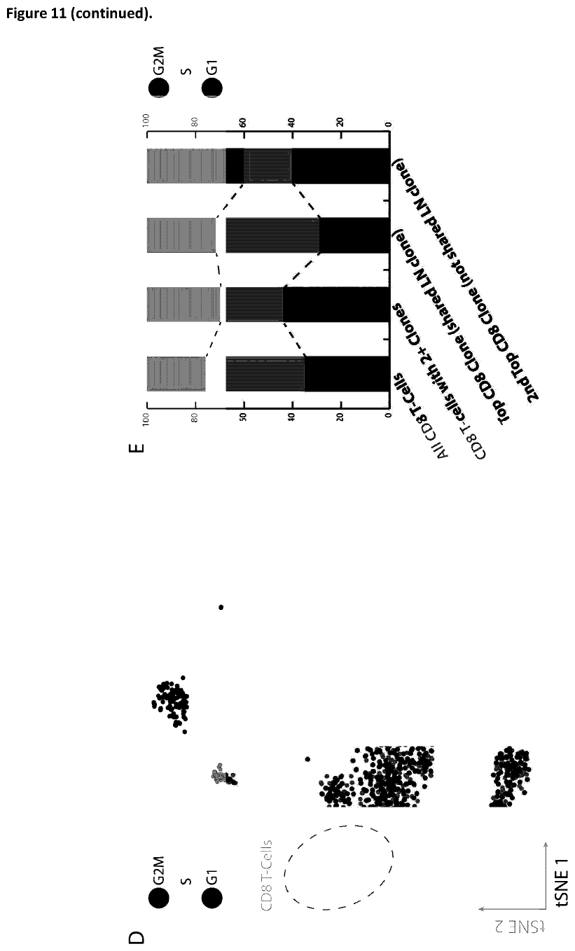

Upon activation, B cells can change their antibody effector function through genome rearrangements in the BCR heavy chain constant region, known as isotype class switching (Di Noia and Neuberger (2007) Annual review of biochemistry, 76:1-22) and also generate membrane-associated or secreted immunoglobulins via alternative splicing (Alt et al., (1980) Cell, 20(2):293-301). Naïve B cells predominantly express BCR transcripts that are non-mutated and express both IGHM and IGHD isotypes. Upon activation, however, B cells mutate their BCR and can replace IGHM with IGHG, IGHE or IGHA isotypes (Alt et al., (1980) Cell, 20(2):293-301); Chaudhuri and Alt (2004) Nature reviews Immunology, 4(7):541-52). Alternative splicing controls the expression of IGHD and IGHM, but also membrane-form and secreted-form transcripts of IgH, which transitions as B cells becoming antibody secreting cells. As expected, memory B cells in the lymph node were more mutated, had undergone isotype switching and had a greater number of IgH clonotypes assigned the secreted-form when compared to naïve B cells (FIG. 9). Surprisingly, up to 30% of naïve B cells were assigned secreted IGHM or secreted IGHD clonotypes. It was also possible to detect the presence of the same IgH clonotype with both membrane and spliced isoforms in a single cell, including IGHD and IGHM isoforms in individual naïve B cells (FIG. 9). Plasmablasts from a tumour from the same patient were also compared (FIG. 11) and it was found that the majority of IgH clonotypes from these cells were assigned IGHA1 isotypes and all assigned the secreted-form only (FIG. 9). This is consistent with plasmablasts being antibody secreting cells, in this case secreting IgA antibodies, common to breast cancer.

Clonal expansion in the lymph node was uncommon, with B or T cell clones only detected in a maximum of two cells within the total population. For B cells there were 13 expanded clones, the majority of which segregated in the naïve B cell cluster, while for T cells there were also 13 expanded clones which clustered by cell type (FIG. 5D). Individual cells belonging to a clone mapped close to each other on the tSNE plot suggesting that cells with the same receptor are more transcriptionally similar to each other. Indeed, B cell and T cell clones with the same receptor sequence present more similar gene expression profiles than non-clonally expanded B cells (P=2.10E-07, paired Wilcoxon test) and T cells (P=2.55E-11) when comparing their Jaccard similarity coefficient for the 250 most abundant genes.

### 1.6 CROSS-TISSUE REPERTOIRE ANALYSIS

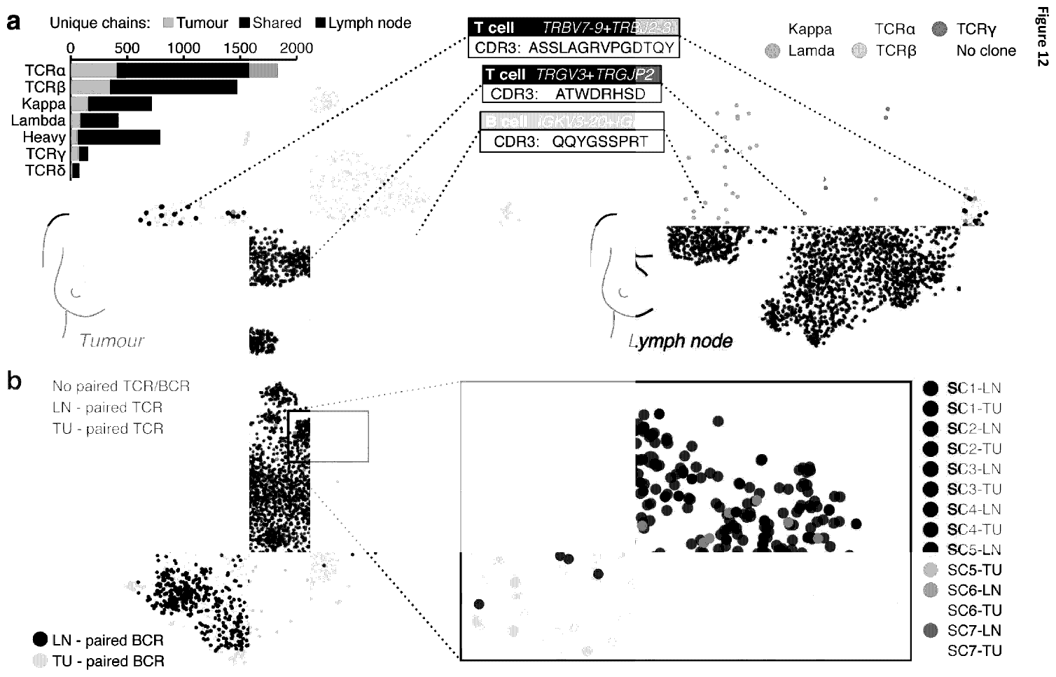

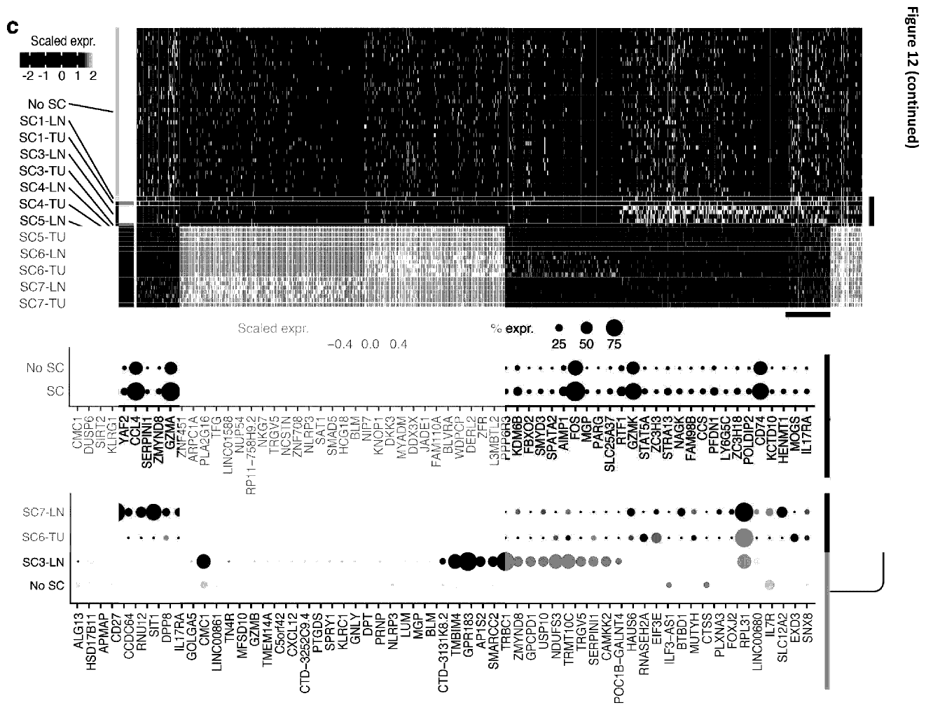

An important application of RAGE-Seq is the ability to track clonally related T or B cells across tissues, to gain systems-level insights into the evolution of immune responses. One such application is the analysis of lymphocytes in a tumour and its draining lymph node, the presumptive site of antigen presentation and source of tumour-infiltrating lymphocytes (TILs). The inventors therefore performed RAGE-Seq on a patient-matched primary tumour and compared the results to the lymphocytes found in the patient's lymph node. From a total of 2493 captured cells, 909 T cells and 215 B cells (FIG. 11) were identified. A substantial number of receptor chains were found to be shared between tissues, specifically 34 light chains, 11 alpha chains, 7 beta chains and 5 gamma chains, present on 157 B-cells and 134 T-cells (FIG. 6A). As expected, the majority of shared single-chains originate from the less diverse secondary chains (IGk, IGl, TCRα, TCRγ). Some chains showed tissue enrichment, with the Immunogloblulin light chain IGLV4-69 IGLJ3 QTWGTGFWV expressed by 27 tumour-resident B-cells and plasmablasts (16.9% of all light chains), but undetected in Lymph node (FIG. 12A).

To investigate whether clonally related cells have common gene expression features across tissues, more stringent thresholds for clonality were applied, analysing lymphocytes expressing paired receptor chains or the highly diverse TCRβ and IGH. Seven shared clones were identified, six of them within the CD8 EFF cluster (FIG. 12B). To allow direct cross-tissue gene expression analysis, canonical correlation analysis (CCA) was used for pairwise integration of the two count matrices generated by each sample. Differential gene expression analysis between shared and non-shared clones revealed a discrete gene signature for the clonally expanded cells compared to non-expanded clones, irrespective of tissue origin (FIG. 12C). Genes highly expressed in active tissue resident cytotoxic lymphocytes such CCL4, NKG7, GZMA, GZMK (Cheuk et al., (2017) Immunity, 21; 46(2):287-300) were common to expanded clones. Interestingly, sets of genes that were uniquely expressed by each of the clones containing 3 or more cells were identified (FIG. 12C).

The presence of clonally expanded T cells between tissues suggested that these cells were proliferating in response to antigen stimulation. To examine this further, the scRNA-Seq data was used to perform cell cycle analysis of all cells within each CD8 (EFF) cluster of tumour and lymph node to infer whether TIL persistence of the clone is through proliferation occurring at the site of each sample and/or through trafficking between tissues. This was additionally performed on any expanded clones in tumour not shared with lymphnode (FIG. 11). A large proportion of T cells were in S, G2 or M phase, suggesting ongoing proliferation.

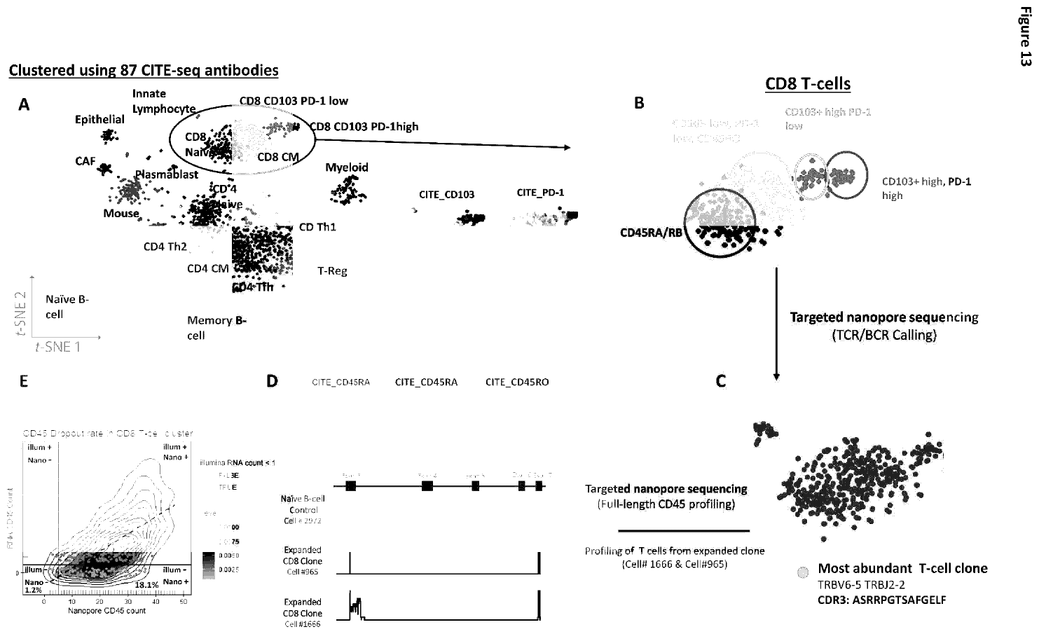

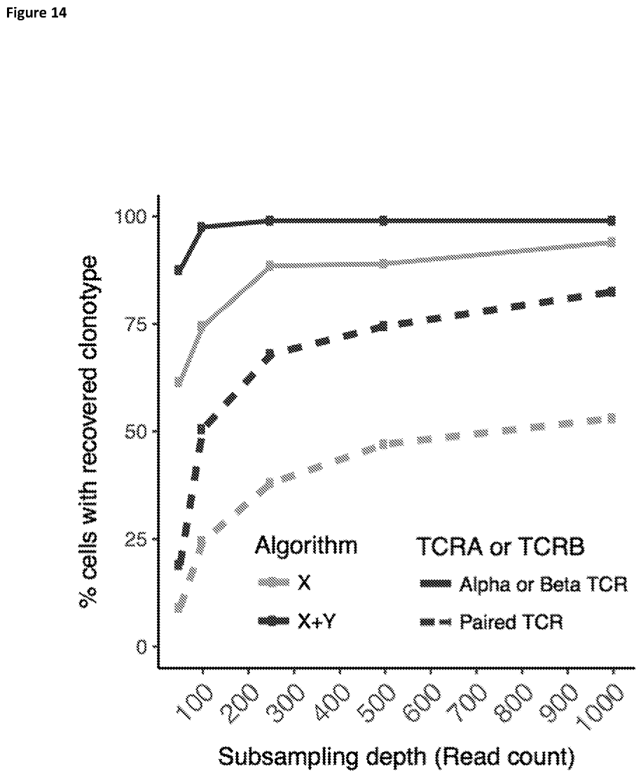

To determine whether RAGE-Seq is compatible with other multi-omic methods, the inventors analysed an additional metastatic lymph node sample (metastatic triple negative breast cancer) using RAGE-Seq together with CITE-Seq, a method to simultaneously determine transcriptome and protein epitope data in thousands of single cells. Cells were stained with a panel of 87 uniquely barcoded antibodies against immune and tumour markers and immune checkpoint molecules and partitioned using the 10× chromium system. A total of 3113 cells were captured using the 10× Chromium platform and subjected to RAGE-Seq, capturing TCR, BCR and PTPRC (the gene encoding CD45). RNA and CITE-Seq libraries were separated by size fractionation and sequenced separately by illumina short-read sequencing while targeted capture long read sequencing using the nanopore platform was conducted for PTPRC (encoding CD45) and all TCR and BCR genes, as described earlier. The data presented in FIG. 13 demonstrates the compatibility of 3-way cDNA, protein and full-length sequencing using a combination of CITE-Seq and RAGE-Seq.

### 1.7 DISCUSSION

Pairing the clonotype of a BCR or TCR with a functional phenotype of a B or T cell offers great insights into B and T cell responses. RAGE-Seq has been shown to be robust in its ability to sample across both Illumina and Nanopore sequencing platforms and highly sensitive and accurate in providing full-length BCR and TCR sequences across immortalized and primary human B and T cells. Given its greater throughput and substantially lower cost, RAGE-Seq has significant advantages over SmartSeq2 for immune profiling. As a result, RAGE-Seq can circumvent the need to isolate specific lymphocyte populations by flow cytometry, permitting retrospective characterization of low abundance lymphocytes within tissues. As shown herein, using RAGE-seq it was possible to identify clones with unique gene expression features that had expanded and were shared across tissues, despite unbiased sampling from a breast cancer, which generally have low TIL frequency.

In this study the inventors demonstrate the compatibility of RAGE-Seq with the 10× Chromium 3′ and/or 5′ system. However, the RAGE-seq pipeline may be adapted to any high-throughput single cell RNA-sequencing technologies that employ 3′ and/or 5′ cell-barcode tagging. Furthermore, a number of these methods are compatible with current DNA barcoded antibody technologies CITE-Seq and REAP-Seq (Stoeckius et al., (2017) Nature methods, 14(9):865-8; Peterson et al., (2017) Nature biotechnology, 35(10):936-9; Shahi et al., (2017) Scientific reports, 7:44447), which are powerful tools for immunophenotyping, allowing the additional measurement of cell surface proteins. The combination of RAGE-Seq with CITE-Seq permits the simultaneous phenotyping of cellular populations using protein targets plus RNA with full length sequencing capacity. This will be hugely valuable to numerous areas of investigation, including tumour immunology, autoimmunity, functional genomics or clonal evolution in cancer. It can be applied more broadly to any study in which the incorporation of feature barcoding, such as CITE-Seq or CRISPR barcodes, adds value to RAGE-Seq. The inventors anticipate that the high nucleotide accuracy achieved by RAGE-Seq will be applicable to identifying somatic variants in individual cancer cells and link this with gene-expression profiles.

Recently, the commercially available Single Cell V(D)J+5′ Gene Expression kit has been used to profile TILs in Breast cancer (Azizi et al., (2018) Cell, 174(5):1293-1308), relying on the incorporation of cell barcodes on the 5′ end of mRNA transcripts and VDJ-specific PCR amplification. Compared to this method, RAGE-Seq has several advantages:

1) it is compatible with CITE-Seq and REAP-Seq and sequences receptors from all lymphocytes in a single reaction, including gd T cells which are of increasing interest in infection and cancer immunology (Zhao et al., (2018) Journal of translational medicine, 16(1):3);

2) RAGE-Seq provides full length receptor sequence, which is essential in the analysis of immunoglobulin SHM; and

3) the recovery of paired full length IgH and IgL sequences also allows for the synthesis of recombinant antibodies which can be used to explore the antigen specificity of B cells of interest.

A further advantage of RAGE-Seq is the ability to detect splice isoforms at the single cell level, which has been demonstrated herein by detecting IgH isoforms destined for antibody secretion or membrane-integration. To the inventors' knowledge, this is the first report integrating IgH V(D)J clonotype sequences with analysis of membrane or secreted exons.

Whilst the focus in this proof of concept study is on lymphocyte receptors, the inventors anticipate that any transcripts could be targeted using this method, simply by changing the composition of the capture probe library. This may include panels targeting cancer driver genes, genes controlling or regulated by splicing, pathogenic fusion genes or genes that are otherwise difficult to detect using short-read sequencing, In this regard, RAGE-seq is a generalisable experimental and computation pipeline to integrate gene expression with targeted analysis of splicing, structural variation and somatic mutation from thousands of single cells. One can envisage this method being applied to multiple areas of biology, such as oncology where RAGE-Seq could be used to track the transcriptional consequences of clonal evolution at single cell resolution. Similarly, RAGE-seq could be applied to neurobiology where alternative splicing and somatic retrotransposition into genes drive brain development and disease (Baillie et al., (2011) Nature, 479(7374):534-7). The adaptability of RAGE-Seq across multiple scRNA-seq platforms and the flexibility to target a range of genes of interest may be of particular value in comprehensively describing a human cell atlas.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method for high-throughput and multiplexed phenotyping and characterisation of single cells, said method comprising:
(a) preparing a library of nucleic acid molecules for one or more isolated single cells, wherein unique cell barcode sequences and unique molecular identifier (UMI) sequences are assigned and introduced to the nucleic acid molecules, optionally wherein unique tissue barcodes are also assigned and introduced to the nucleic acid molecules;
(b) dividing the library into at least two components comprising a first library component and a second library component;
(c) sequencing the first library component to produce a first set of sequence data;
(d) high-throughput molecular profiling of the first set of sequence data to identify sequences containing genetic, epigenetic and/or transcriptomic features that are capable of distinguishing between different cells;
(e) sequencing the second library component using a long-read sequencing method to produce a second set of sequence data comprising long-read sequences;
(f) demultiplexing the second set of sequence data to distinguish between individual long-read sequences;
(g) inferring molecular profiles for the demultiplexed long-read sequences based on molecular profiles characterised for corresponding sequences in the first set of sequence data at (d), wherein corresponding sequences are identified using the UMIs, unique cell barcodes, unique tissue barcodes, or a combinations thereof;
(h) assigning the long-read sequences into one or more groups based on information relating to one or more of tissue type, cell type, genes, sequences and/or molecules of interest, and generating one or more contigs based on consensus sequences identified within the one or more groups; and
(h) undertaking molecular characterisation of the contigs.

2. The method of claim 1, further comprising a single cell capture step prior to step (a).

3. The method of claim 2, wherein the single cell capture step comprises single cell capture by one or more of the following means: a droplet-based microfluidics platform, a flow cytometry platform, a plate-based platform, microwell-based platform or any combination thereof.

4. The method of claim 2 or 3, further comprising isolating the cells prior to the single cell capture step by disassociating tissue or bodily fluid into cellular components, or by selection of one or more subsets of cells from said tissue or bodily fluid.

5. The method of any one of claims 1 to 4, wherein the library of nucleic acid molecules prepared at (a) comprises one or more types of nucleic acid molecule selected from the group consisting of cDNA, genomic DNA, barcodes, cellular RNA and combinations thereof.

6. The method of any one of claims 1 to 5, wherein the library of nucleic acid molecules prepared at (a) is a library of cDNA molecules.

7. The method according to any one of claims 1 to 6, wherein the first library component is sequenced using a short-read sequencing method and/or a long-read sequencing method.

8. The method according to claim 7, wherein the short-read sequencing method is a next generation sequencing (NGS) method selected from the group consisting of sequencing-by-hybridization, sequencing-by-synthesis, sequencing-by-ligation platform, ion semiconductor sequencing, combinatorial probe anchor synthesis sequencing and combinations thereof.

9. The method according to any one of claims 1 to 8, wherein the long-read sequencing method is selected from a nanopore sequencing method, a single molecule real time (SMRT) sequencing method or combinations thereof.

10. The method according to any one of claims 1 to 9, comprising targeted enrichment of the first and/or second library components for sequences or features of interest prior to sequencing and/or in silico post-sequencing.

11. The method of claim 10, wherein the targeted enrichment is performed prior to sequencing using a hybridisation capture protocol.

12. The method of claim 11, wherein the hybridisation capture protocol relies on biotinylated hybridisation beads attached to capture probes which bind selectively to genetic, epigenetic or transcriptomic sequences or features or interest within the library component(s).

13. The method according to any one of claims 1 to 12, comprising targeted enrichment of the first and/or second library components by depleting unwanted sequences or features from the library component(s) prior to sequencing and/or depleting unwanted sequences or features from the sequence data in silico.

14. The method according to any one of claims 10 to 13, wherein the targeted enrichment is for T and/or B cell receptor sequences and/or immunomodulatory genes.

15. The method according to any one of claims 1 to 14, wherein molecular characterisation of the contigs comprises characterisation on the basis of one or more of the following: antigen receptor clonotyping, mutation analysis, somatic genome variation, alternative transcript splicing, fusion genes or chimeric transcripts, transcript isoform quantification and combinations thereof.

16. The method according to any one of claims 1 to 15, wherein the molecular characterisation of the contigs comprises characterisation on the basis of any one or more of the following:
(i) information from (d) inferred from the corresponding sequences in the first set of sequence data;
(ii) information relating to target enrichment for sequences or features of interest performed on the second library component prior to sequencing and/or in silico following sequencing;
(iii) alignment of long-read sequences or contigs to an annotated reference sequences or genomes; and/or
(iv) information relating to the one or more of the unique cell barcodes, UMI sequences and/or unique tissue barcodes.

17. The method according to any one of claims 1 to 16, comprising performing one or more filtering steps on the second set of sequence data to remove sequences which are below a desired length, uninformative, erroneous and/or not of interest.

18. The method according to any one of claims 1 to 17, wherein demultiplexing the second set of sequence data is supervised.

19. The method according to any one of claims 1 to 17, wherein demultiplexing the second set of sequence data is unsupervised.

20. The method of claim 19, wherein:
(i) supervised demultiplexing comprises comparing or matching the long-read sequences to the corresponding sequences in the first set of sequence data using the UMIs, unique cell barcodes, unique tissue barcodes or combinations thereof; and/or
(ii) unsupervised demultiplexing comprises comparing the second set of sequence data to itself in a manner to identify commonalities in long-read sequences or identifying sequence features selected from UMI, unique cell barcodes and/or unique tissue barcodes.

21. The method according to any one of claims 1 to 20, wherein assignment of the demultiplexed long-read sequences into one or more groups comprises de novo assembly of long-read sequences, alignment to one or more reference sequences, multiple sequence alignments or other approach capable of grouping the long-read sequences.

22. The method according to any one of claims 1 to 21, comprising one or more step to correct errors in the long-read sequences and/or contigs to improve the consensus sequences.

23. A computer implemented method for phenotyping and characterising single cells using data obtained from high-throughput and multiplexed long-read single cell sequencing, said method comprising:
(a) receiving a first set of sequence data for a library of nucleic acid molecules generated for one or more isolated single cells, wherein each nucleic acid molecule in the library comprises a unique cell barcode sequence and unique molecular identifier (UMI) sequence, optionally wherein each nucleic acid molecule in the library also comprises a unique tissue barcode;
(b) high-throughput molecular profiling of the first set of sequence data by identifying sequences containing genetic, epigenetic and/or transcriptomic features that are capable of distinguishing between different cells;
(c) receiving a second set of sequence data for the library of nucleic acid molecules, said second set of data comprising long-read sequences;
(d) demultiplexing the second set of sequence data to distinguish between individual long-read sequences;
(e) inferring molecular profiles for the demultiplexed long-read sequences based on molecular profiles characterised for corresponding sequences in the first set of sequence data at (b);
(f) assigning the long-read sequences into one or more groups based on information relating to one or more of tissue type, cell type, genes, sequences and/or molecules of interest and generating one or more contigs based on consensus sequences identified within the one or more groups;
(g) undertaking molecular characterisation of the contigs; and
(h) generating user interface data comprising information relating to molecular characterisation of the contigs.

24. The method of claim 23, wherein the library of nucleic acid molecules comprises one or more types of nucleic acid molecule selected from the group consisting of cDNA, genomic DNA, barcodes, cellular RNA and combinations thereof.

25. The method of claim 23 or 24, wherein the library of nucleic acid molecules is a library of cDNA molecules.

26. The computer implemented method of any one of claims 23 to 25, wherein the first set of sequence data is generated by a short-read sequencing method and/or a long-read sequencing method.

27. The computer implemented method according to claim 26, wherein the short-read sequencing method is a next generation sequencing (NGS) method selected from the group consisting of sequencing-by-hybridization, sequencing-by-synthesis, sequencing-by-ligation platform, ion semiconductor sequencing, combinatorial probe anchor synthesis sequencing and combinations thereof.

28. The computer implemented method according to any one of claims 23 to 26, wherein the second set of sequence data is generated by a nanopore sequencing method or a single molecule real time (SMRT) sequencing method.

29. The computer implemented method according to any one of claims 23 to 28, wherein the first and/or second set of sequence data is enriched for genetic, epigenetic or transcriptomic sequences or features or interest.

30. The computer implemented method according to any one of claims 23 to 29, wherein the enrichment occurred prior to sequencing or is performed on the sequence data in silico.

31. The computer implemented method according to any one of claims 23 to 30, wherein the targeted enrichment includes a step of depleting unwanted sequences or features from the library of nucleic acid molecules prior to sequencing and/or depleting unwanted sequences or features from the sequence data in silico.

32. The computer implemented method according to any one of claims 29 to 31, wherein the targeted enrichment is for T and/or B cell receptor sequences and/or immunological gene sequences.

33. The computer implemented method according to any one of claims 23 to 32, wherein molecular characterisation of the contigs comprises characterisation on the basis of one or more of the following: antigen receptor clonotyping, mutation analysis, somatic genome variation, alternative transcript splicing, fusion genes or chimeric transcripts, transcript isoform quantification and combinations thereof.

34. The computer implemented method according to any one of claims 23 to 33, wherein molecular characterisation of the contigs comprises characterisation on the basis of one or more of the following:
(i) information relating to the molecular profile of long-read sequences inferred at (e);
(ii) information relating to target enrichment for sequences or features of interest;
(iii) alignment of long-read sequences or contigs to an annotated reference sequences or genomes; and/or
(iv) information relating to the one or more of the unique cell barcodes, UMI sequences and/or unique tissue barcodes.

35. The computer implemented method according to any one of claims 23 to 34, comprising performing one or more filtering step on the second set of sequence data to remove sequences which are below a desired length, uninformative, erroneous and/or not of interest.

36. The computer implemented method according to any one of claims 23 to 35, wherein demultiplexing the second set of sequence data is supervised.

37. The computer implemented method according to any one of claims 23 to 36, wherein demultiplexing the second set of sequence data is unsupervised.

38. The computer implemented method of claim 37, wherein:
(i) wherein supervised demultiplexing comprises comparing or matching the long-read sequences to the corresponding sequences in the first set of sequence data using the UMIs, unique cell barcodes, unique tissue barcodes or combinations thereof; and/or
(ii) unsupervised demultiplexing comprises comparing the second set of sequence data to itself in a manner to identify commonalities in long-read sequences or identifying sequence features selected from UMI, unique cell barcodes and/or unique tissue barcodes.

39. The computer implemented method according to any one of claims 23 to 38, wherein assigning the demultiplexed long-read sequences into one or more groups comprises de novo assembly of long-read sequences, alignment to one or more reference sequences, multiple sequence alignments or other approach capable of grouping the long-read sequences.

40. The computer implemented method according to any one of claims 23 to 39, comprising one or more steps to correct errors in the long-read sequences and/or contigs to improve the consensus sequence.
